(19) United States
(12) Patent Application Publication
(10) Pub. No.: US 2019/0330297 A1
(43) Pub. Date: Oct. 31, 2019

(54) INSULIN GENE-DERIVED PROTEINS AND PEPTIDES FOR USE IN THE DIAGNOSIS AND TREATMENT OF TYPE 1 DIABETES

(71) Applicant: ACADEMISCH ZIEKENHUIS LEIDEN, Leiden (NL)

(72) Inventors: Bart Otto ROEP, Leiden (NL); Menno VAN LUMMEL, Leiden (NL); Peter Antonis VAN VEELEN, Leiden (NL); Arnaud ZALDUMBIDE, Leiden (NL)

(21) Appl. No.: 16/071,900

(22) PCT Filed: Jan. 20, 2017
(86) PCT No.: PCT/EP2017/051247

§ 371 Date: Jul. 20, 2018

(30) Foreign Application Priority Data
Jan. 22, 2016 (EP) 16152400.4
Feb. 4, 2016 (EP) 16154295.6

(51) Int. Cl. C07K 14/62; A61K 35/15; C07K 16/26; G01N 33/68; G01N 33/569

(52) U.S. Cl. CPC C07K 14/62; A61K 35/15; A61K 38/00; G01N 33/6893; G01N 33/56972; C07K 16/26

(57) ABSTRACT

The present invention relates to the identification of novel peptides derived from aberrant proteins involved in Type 1 Diabetes mellitus (T1D), also known as defective ribosomal products (DRiPs). In particular the invention relates to epitopes present in DRiPs of the human preproinsulin (PPI) mRNA, its representative peptides and the use thereof in diagnosis, prevention and/or treatment of T1D. Moreover, the inventions relate to antibodies and antisera against the identified epitopes and the use thereof in the diagnosis of T1D.

### FIELD OF THE INVENTION

The invention relates to the field of medicine. In particular, the invention relates to the field of autoimmune diseases and specifically to type 1 diabetes. The invention also relates to non-conventional β-cell polypeptides and their role in type 1 diabetes mellitus. Disclosed are novel T-cell epitopes and their use in the treatment, prevention and diagnosis of type 1 diabetes mellitus.

### BACKGROUND OF THE INVENTION

Type 1 diabetes mellitus (herein also referred to as type 1 diabetes, or T1D) is an autoimmune disorder wherein the immune system inadvertently and progressively destroys the β-cells (the insulin-producing cells) in the pancreas. T1D is characterized by a loss of immune tolerance to the β-cells, while neighboring α- and δ-cells are spared. In this process, it is believed that CD8+ and CD4+ T-lymphocytes (generally and herein also referred to as T-cells) are important players in T1D pathogenesis. In addition, it has extensively been documented that autoantibodies are being raised against several proteins that are presented by β-cells, which is indicative for the loss of tolerance against self-proteins. The 65 kD isoform of glutamic acid decarboxylase (GAD65), the insulinoma-associated-2 protein (IA-2, sometimes also referred to as Islet Tyrosine Phosphatase-like protein) and preproinsulin (PPI) are examples of such autoantigens. Epitope identification is important for developing diagnostic and therapeutic tools for immune-mediated diseases (Collins E J and Frelinger J A 1998. Altered peptide ligand design: altering immune responses to class I MHC/peptide complexes. Immunol Rev 163:151-160; Davenport M P and Hill A V 1996. Reverse immunogenetics: from HLA-disease associations to vaccine candidates. Mol Med Today 2:38-45; van Lummel, Diabetes, 2015, pii:db151031). The MHC haplotypes highly associated with T1D development are well known (Concannon et al. NEJM, 2009; Koeleman et al. Genes Immun, 2004). The autoimmune attack on β-cells in the pancreas proceeds by way of the MHC class II (and MHC class I) pathway, in which antigen presenting cells (APCs) take up and process relevant β-cell protein autoantigens and present their peptide epitopes to CD4+ and CD8+ T-cells, thereby inducing cytokines which assist in the destruction of β-cells. Therapeutic intervention trials in patients with T1D and those at risk of the disease are fast becoming a major focus for clinical research and the need for disease biomarkers that represent islet-damaging or islet-protective events, has become more urgent (Mallone et al. Clin Immunol, 2013). Also, it was realized that β-cell specific peptides could be applicable in immunotherapy against T1D (Lernmark et al. Nat Rev Endocrinol, 2013). Hence, several investigators have identified peptides that are be recognized by T-cells (Mallone et al. Clin Dev Immunol, 2011).

In peptide immunotherapy delivery of soluble native peptide leads to the generation/expansion of specialized CD4+ T-cells that are regulatory. Regulatory T-cells are capable of specifically inhibiting the damaging of islet cells by release of anti-inflammatory cytokines, for example interleukin-10 (IL-10). Regulatory T-cells that operate through release of IL-10 are termed Tr1 cells. Induction of Tr1 cells through peptide immunotherapy is one of the very few therapeutic approaches to offer an outcome in which immunological tolerance to β-cells is restored. The use of peptides over using the whole antigen has several advantages: peptides are easy to produce, relatively easy to pharmaceutically formulate and to quality assure. They do not carry the biological side-effects of the parent protein. Several T-cell epitopes that appear specific for T1D have been discovered and have been described in the art (WO 2009/004315; WO 01/13934; U.S. Pat. No. 7,049,292). Peptides have been found after vaccination in mice, by screening of libraries of synthetic peptides with arbitrary length, or by elution of naturally processed and presented epitopes (NPPEs) from HLA-DR4 (Peakman et al. JCI, 1999; Arif et al. JCI, 2004) of pulsed B lymphocytes and recently from HLA-DQ2 and DQ8 of pulsed dendritic cells (DCs; van Lummel et al Diabetes, 2015). Although several reports have disclosed the discovery of peptides that could be presented by APCs derived from autoantigens present in β-cells of the pancreas, to date no therapies to treat T1D in patients using peptide immunotherapy have been initiated. Several disadvantages of the identified peptides in the art still exist. Peptides that were identified using synthetic peptide libraries have the downside that these peptides do not represent what peptides may be naturally presented in real life. Such peptides may lack the boundary sequences required for proper presentation and recognition by T cells, or may be processed in non-natural ways within the APC. There is an ongoing need for novel peptides that are naturally processed and presented, most preferably processed by APC's that occur in nature.

### SUMMARY OF THE INVENTION

The present invention relates to a polypeptide or peptide comprising an epitope present in a Defective Ribosomal Product (DRiP) from the human PPI mRNA. Preferably, said DRiP is translated from an out-of-frame ATG start codon within said human PPI mRNA and even more preferably, said DRiP comprises the amino acid sequence of SEQ ID NO:3 or SEQ ID NO:4. Preferably, the epitope comprises at least 5 consecutive amino acids present in said DRiP and even more preferably, the epitope comprises an amino acid sequence selected from the group consisting of LHRERWNKALEPAK (SEQ ID NO: 7) and MLYQHLLPL (SEQ ID NO: 8). Preferably, the epitope is a T-cell epitope and even more preferably, said T-cell epitope comprises the amino acid sequence MLYQHLLPL (SEQ ID NO: 8). In one embodiment, the polypeptide or peptide is isolated.

The present invention also relates to a polypeptide or peptide according to the invention and/or a cytotoxic T-cell targeting said polypeptide or peptide for use in the diagnosis of type 1 diabetes mellitus.

The present invention also relates to use of a polypeptide or peptide according to the invention and/or a cytotoxic T-cell targeting said polypeptide or peptide, as a biomarker for type 1 diabetes mellitus and/or in detecting β-cell stress.

The present invention also relates to an isolated nucleic acid encoding a polypeptide or peptide according to the invention or the complementary nucleic acid thereof.

The present invention also relates to an expression vector comprising a nucleic acid encoding a polypeptide or peptide as defined in any one of claims 1 to 8.

The present invention also relates to an isolated peptide-MHC complex, wherein the peptide forming part of the complex is a polypeptide or peptide according to the invention.

The present invention also relates to an isolated binding molecule capable of specifically binding a peptide-MHC complex according to the invention or a polypeptide or a peptide according to the invention. In one embodiment, the isolated binding molecule is an isolated antibody, capable of specifically binding a polypeptide or peptide according to the invention. In another embodiment, the isolated binding molecule comprises complementarity determining regions derived from a TCR capable of specifically binding a peptide-MHC according to the invention.

The present invention also relates to a polyclonal antiserum raised and/or directed against a polypeptide or peptide according to the present invention. Said polyclonal antibody can for instance be used in the diagnosis of type 1 diabetes mellitus.

The present invention also relates to use of an isolated binding molecule according to the invention in in vivo and in vitro imaging, for drug targeting, in the diagnosis of type 1 diabetes mellitus or in detecting β-cell stress.

The present invention also relates to a method for identifying a subject suffering from, or being at risk of suffering from, type 1 diabetes mellitus, the method comprising:
a) measuring the amount of polypeptide or peptide according to the invention a blood, serum or plasma sample from said subject; and
b) comparing the measured amount of said polypeptide or peptide to a reference value,

wherein a significant deviation in the amount of measured polypeptide or peptide compared to the reference value, is indicative of type 1 diabetes mellitus. Preferably this method comprises using an isolated antibody according to the invention or a polyclonal antiserum according to the invention for measuring the amount of said polypeptide or peptide.

The present invention also relates to a method for identifying a subject suffering from, or being at risk of suffering from, type 1 diabetes mellitus, the method comprising:
a) measuring the amount of cytotoxic T-cells targeting a polypeptide or peptide according to the invention in a blood sample from said subject; and
b) comparing the measured amount of said cytotoxic T-cells to a reference value,

wherein a significant deviation in the amount of measured cytotoxic T-cells compared to the reference value, is indicative of type 1 diabetes mellitus. Preferably, this method comprises using an isolated peptide-MHC complex according to the invention for measuring the amount of said cytotoxic T-cells.

The present invention also relates to a culture of tolerance promoting cells targeting a polypeptide or peptide according to the invention. Preferably, said tolerance promoting cells are selected from the group consisting of immature dendritic cells and dendritic cells.

The present invention also relates to a composition comprising an isolated polypeptide or peptide according to the invention. In one embodiment, said composition further comprises a culture of tolerance promoting cells according to the invention. In another embodiment, said composition further comprises a tolerance promoting adjuvant. Preferably, a composition according to the invention is a pharmaceutical composition.

The present invention also relates to a composition according to the invention for use as a medicament. Preferably, the present invention relates to a composition according to the invention, for use in a method for the prevention and/or treatment of type 1 diabetes mellitus. Preferably, the present invention relates to a composition according to the invention for the prevention and/or treatment of type 1 diabetes mellitus, wherein said composition is administered at an interval of from about 20 to about 36 days.

The present invention also relates to use of an isolated polypeptide or peptide according to the invention, an isolated nucleic acid according to the invention, an isolated peptide-MHC complex according to the invention, an isolated binding molecule according to the invention or a polyclonal antiserum according to the invention for drug targeting.

The present invention also relates to use of an isolated polypeptide or peptide according to the invention, an isolated nucleic acid according to the invention, an isolated peptide-MHC complex according to the invention, an isolated binding molecule according to the invention or a polyclonal antiserum according to the invention for isolation, purification and/or quantification of a binding partner or a cell comprising a binding partner.

The present invention also relates to use of an isolated polypeptide or peptide according to the invention, an isolated nucleic acid according to the invention, an isolated peptide-MHC complex according to the invention, an isolated binding molecule according to the invention or a polyclonal antiserum according to the invention in in vitro and in vivo imaging.

The present invention also relates to use of an isolated polypeptide or peptide according to the invention, an isolated nucleic acid according to the invention, an isolated peptide-MHC complex according to the invention, an isolated binding molecule according to the invention or a polyclonal antiserum according to the invention in the diagnosis of type 1 diabetes mellitus.

The present invention also relates to use of an isolated polypeptide or peptide according to the invention, an isolated nucleic acid according to the invention, an isolated peptide-MHC complex according to the invention, an isolated binding molecule according to the invention or a polyclonal antiserum according to the invention in detecting β-cell stress.

The present invention also relates to use of an isolated polypeptide or peptide according to the invention, an isolated nucleic acid according to the invention, an isolated peptide-MHC complex according to the invention, an isolated binding molecule according to the invention, a polyclonal antiserum according to the invention, a culture of tolerance promoting cells according to the invention or a composition according to the invention for the prevention and/or treatment of type 1 diabetes mellitus.

The present invention also relates to an isolated polypeptide or peptide according to the invention, an isolated nucleic acid according to the invention, an isolated peptide-MHC complex according to the invention, an isolated binding molecule according to the invention or a polyclonal antiserum according to the invention for use in the diagnosis of type 1 diabetes mellitus.

The present invention also relates to an isolated polypeptide or peptide according to the invention, an isolated nucleic acid according to the invention, an isolated peptide-MHC complex as according to the invention, an isolated binding molecule according to the invention, a polyclonal antiserum according to the invention, a culture of tolerance promoting cells according to the invention or a composition according to the invention for use as a medicament.

The present invention also relates to an isolated polypeptide or peptide according to the invention, an isolated nucleic acid according to the invention, an isolated peptide-MHC complex according to the invention, an isolated binding molecule according to the invention, a polyclonal antiserum according to the invention, a culture of tolerance promoting cells according to the invention or a composition according to the invention for use in the prevention and/or treatment of type 1 diabetes mellitus.

The present invention also relates to a method of treating a human subject having type 1 diabetes mellitus, or that is at risk of developing type 1 diabetes mellitus, said method comprising administering to said human subject an effective amount of an isolated polypeptide or peptide according to the invention, an isolated nucleic acid according to the invention, an isolated peptide-MHC complex according to the invention, an isolated binding molecule according to the invention, a polyclonal antiserum according to the invention, a culture of tolerance promoting cells according to the invention or a composition according to the invention.

### BRIEF DESCRIPTION OF THE DRAWINGS

FIG. 1 shows the DNA sequence (SEQ ID NO:1) of the wild type human insulin gene in lower case letters. The insulin gene encodes the gene product PPI and is therefore sometimes referred to as the PPI gene. The translated amino acid sequence (SEQ ID NO:2) of the wild type human insulin gene with its normal start at the first occurring ATG codon. This 110 amino acid sequence is the human wild type in-frame sequence of PPI and is shown in bold capitals. The 43 amino acid DRiP that is translated from the out of frame ATG start codon shared between codon 94 and 95 within the normal coding sequence is provided in italic capitals (also referred to as INS-DRiP polypeptide herein). Two SNPs rs3842752 and rs3842753 (Marchand et al., Diabetes, 2007) have previously been shown to be associated with T1D. These two SNPs are located in the 3′UTR region of the insulin gene. In case of alternative initiation two polypeptides variants would be generated, namely INS-DRiP-RP and INS-DRiP-CH (here the INS-DRiP-RP sequence is shown). Both polypeptides were found to be present in nature as shown herein. The CD8 and CD4 T cell epitope that was identified by the methods of the present invention are the first 9 amino acids from the N-terminus of the—INS-DRiP: MLYQHLLPL (SEQ ID NO: 8).

FIG. 2 shows the full-length PPI mRNA sequence in the first row. The PPI mRNA sequence comprises 5′UTR (nucleotide no. 1-59), the normal in-frame open reading frame (ORF) (nucleotide 60-389), 3′-UTR (nucleotide no. 390-469) and the poly-A signal sequence (nucleotide no. 390-469). The amino acid sequence of PPI is shown in the second row, starting at nucleotide no. 60 and ending at the stop codon starting at nucleotide no. 390. The amino acid sequence of the +2 out of frame ORF is shown in the third row starting at nucleotide 2. INS-DRiP is translated from this +2 out of frame ORF and the amino acid sequence of INS-DRiP is depicted in bold starting at nucleotide no. 341. The two SNPs, rs3842752 and rs3842753, are marked in the 3′-UTR. The two SNPs are annotated and both polymorphisms are depicted together with the potentially affected amino acids in the non-conventional INS-DRiP polypeptide. All AUG (start) codons within the mRNA are framed. The * indicate the stop codons in the given amino acid sequence. Putative non AUG (CUG) start codons upstream the INS-DRiP sequence is framed with a dashed line.

FIG. 3A shows a schematic overview of the full-length human PPI mRNA. The PPI open reading frame (ORF) starts at AUG at position 60. Three alternative translation initiation sites are located at position 72, 341 and 442. The poly-A tail is indicated. The ORFs of the insulin DRiPs of the present invention start at the AUG at position 341. The table on the right indicates the translation initiation score of every AUG within the insulin mRNA sequence, as predicted by NetStart 1.0 prediction server. Predictions scores greater than 0.5 are considered probable translation start codons. The AUG at position 442 was excluded from putative translation initiation due to the low prediction score (asterisks).

FIG. 3B shows a schematic representation of the position where the INS-DRiP translation starts within the PPI mRNA.

FIG. 3C shows a schematic representation of the position where the splice variant of PPI is generated.

FIG. 4A shows as schematic representation of the GFP fusion constructs made. INS-GFP is a fusion product where the GFP coding sequence is cloned in-frame with the insulin gene (starting at AUG60). INS-DRiP-GFP is a fusion product where the GFP coding sequence is cloned in-frame with the DRiP gene (starting at AUG341). DRiP-GFP is a control fusion product, where the DRiP sequence alone is fused to GFP. The expected sizes of the fusion constructs are depicted on the right.

FIG. 4B is a western blot using anti-GFP antibody (top panel) or anti-actin antibody used as loading control (lower panel) on lysates of HEK 293T cells transfected with GFP fusion constructs. In the left panel the following constructs are shown: GFP control, INS-GFP, and DRiP-GFP (expressed from the control construct CMV-DRiP-GFP) showing that the expressed GFP fusion proteins migrates at 40 kDa and 33 kDa, respectively. The right panel shows the INS-DRiP-GFP control construct. It is seen that the molecular weight of the unconventional product INS-DRiP-GFP was confirmed to be the same as for the DRiP-GFP control.

FIG. 4C is a western blot of cells transfected with INS-DRiP-GFP, showing co-expression of proinsulin.

FIG. 5 shows the effect of thapsigargin (TG) and tunicamycin (TM) on the expression level of INS-DRiP-GFP as quantified both by western blot (FIG. 5, upper panel) and flow cytometry analysis (FIG. 5, lower panel). TG and TM induce endoplasmic reticulum stress (ER stress) by two different mechanisms. The expression of DRiP polypeptide was increased by TG treatment as quantified both by western blot (FIG. 5, upper panel) and flow cytometry analysis (FIG. 5, lower panel). These data indicate that ER stress contributes to expression of the DRiP polypeptide similarly as to other stress-induced proteins.

FIG. 6A shows the T cell responses within peripheral blood mononuclear cells (PBMCs) isolated from T1D patients against the aberrant INS-DRiP-CH and INS-DRiP-RP proteins. Proliferative responses in presence of the INS-DRiP-CH or INS-DRiP-RP recombinant proteins is shown as the stimulation index (SI) calculated by dividing the mean CPM in presence of protein by the mean CPM of medium alone; SI≥3 are considered as positive. A strong T-cell proliferative responses against the INS-DRiP-CH and INS-DRiP-RP proteins were observed in T1D patients carrying the high-risk INS-DRiP-RP variant (left), whereas patients carrying the protective DR15/DQ6 haplotype or the protective INS-DRiP-CH variant responded only to the INS-DRiP-CH protein (right). Patients heterozygous for DQ2 and DQ8 (highest susceptible risk for T1D) show higher proliferative responses to the DRiP variants as compared to patients carrying either DQ2 or DQ8.

FIG. 6B shows proliferative responses against INS-DRiP variant of PBMC isolated from T1D patients carrying the highest risk HLA-DQ2/8 genotype (DQ2/DQ8) compared to patients carrying DQ2/x or DQ8/x and compared to patients carrying neither HLA-DQ2 nor HLA-DQ8 (x/x).

FIG. 6C shows HLA-DQ peptidome analysis of dendritic cells (DCs). Immature DCs, heterozygous for HLA-DQ2/8 were pulsed with the DRiP proteins. After maturation, HLA-DQ molecules were purified from the lysed pulsed DCs, whereafter the naturally processed and HLA bound peptides were acid eluted. Using state-of-the-art mass spectrometry (van Lummel, Diabetes, 2015) a peptide with the sequence MLYQHLLPL derived from the N-terminus of the DRiP variants was identified. The experimental spectrum (top) of this MLYQHLLPL peptide, now identified as a naturally processed peptide eluted from highest-risk HLA-DQ2/8 heterozygous DCs, fully matches the spectrum of the same synthesized peptide (bottom).

FIG. 6D shows binding of the MLYQHLLPL peptide to HLA-DQ. Binding of the MLYQHLLPL peptide was confirmed in cell-free HLA-DQ peptide binding assays (van Lummel, J B C, 2011). Binding of MLYQHLLPL was observed for the T1D highest-risk DQ8cis and DQ8trans molecules. Binding of MLYQHLLPL to T1D low risk DQ2cis/trans molecules was not observed.

FIG. 7 shows the CD8 response to MLYQHLLPL.

FIG. 7A shows the frequency of MLYQHLLPL-specific CTLs (left) and virus-specific CTLs (right) in healthy individuals (HC) and T1D patients (T1D). P values were calculated by unpaired Student's t-test; wherein ns=not significant. Significantly higher levels of MLYQHLLPL-specific CTLs in T1D patients were detected whereas antiviral CTL frequencies were similar between T1D patients and healthy donors.

FIG. 7B: A T-cell clone was generated from PBMCs isolated from a long-term HLA-A2+ T1D patient. After 28 days in culture with periodic peptide specific re-stimulation, CD8+ Tm+ cells were detected (upper panel), flow-sorted and seeded 1 cell/well. MLYQHLLPL-directed CTL clones were validated by dual tetramer (using APC and PE as fluorescent labels) staining (lower panel).

FIG. 7C shows the specific lysis of MLYQHLLPL-loaded JY cells. JY cells were incubated overnight with the MLYQHLLPL-specific CTLs generated as described in FIG. 7B and the specific lysis of MLYQHLLPL-loaded JY cells and irrelevant peptide (SP-PPi₁₅₋₂₄) loaded JY cells is shown. Complete lysis of JY cells loaded with the MLYQHLLPL peptide was detected (black squares), while JY cells loaded with a irrelevant peptide remain unaffected (open circles). Each E:T ratio was measured in triplicate.

FIG. 7D shows lysis of JY cells. Although the isolated clones have different TCR sequences, complete lysis of peptide-pulsed target JY cells was detected (grey filled circles and squares), whereas target cells loaded with irrelevant peptide remained unaffected (black filled circles and squares).

FIG. 8 shows the cytokine production by MLYQHLLPL-directed CTLs after co-culture with MLYQHLLPL-loaded JY cells. P values were calculated by unpaired Student's t-test. Peptide-specific T-cell activation resulted in a significant increased secretion of the proinflammatory cytokines IFNγ and TNFα and to a lesser extent MIP-1β when compared to non-stimulated T-cells.

FIG. 9A shows .percentage of β-cell death (% of decreased luciferase) after 48 h after co-culture with PPi₁₅₋₂₄-directed CTLs (open triangles), pp65CMV directed CTLs (open circles) or MLYQHLLPL-directed CTLs (black squares) in normal low glucose media (5 mM) (left) and inflammatory condition (20 mM glucose, IFN-γ and IL1β) (right), respectively. P values were calculated by unpaired Student's t-test relative to pp65CMV directed CTLs. Under homeostatic conditions PPI₁₅₋₂₄-directed and MLYQHLLPL-directed CTLs significantly reduced beta cell survival, whereas beta cell survival was unaffected by CMV-directed CTLs (left panel). Inflammatory conditions further increased beta cell death in the presence of MLYQHLLPL-directed CTL (right panel).

FIG. 9B shows percentage of β-cell death observed in two different pancreas preparation after incubation with MLYQHLLPL-specific CTL under homeostatic or inflamed conditions (E:I ratio 1:5). P values were calculated by unpaired Student's t-test relative to homeostatic condition. These results indicate that INS-DRiP epitope presentation is enhanced under inflammatory conditions.

FIG. 9C shows cytokine production by MLYQHLLPL-directed CTLs after co-culture with HLA-A2+ human islet cells. IFN-γ, TNF-α and MIP-1β were measured after 48 h incubation in low glucose culture conditions. P value was calculated by unpaired Student's t-test relative to cytokines released by islets in absence of T-cells (IFN-γ and TNF-α) or cytokines released by CTL in absence of islets (MIP-1β).

FIG. 10 shows a western blot of HEK 293T cells transfected with the constructs encoding the normal insulin products or the aberrant insulin DRiP. Total cell lysate was loaded on SDS page and protein was stained with anti-insulin showing a product of 11 kDa (left) or anti-DRiP/Splice antibody showing a product of 9 kDa (right). Although the Poab does not discriminate between the DRiP variants and the Splice variant, these results show that the human insulin gene encodes the INS-DRiP polypeptide, which is a frameshift translation product.

### DETAILED DESCRIPTION

Islet autoreactive T-cells selectively and progressively destroy the insulin-producing β-cells in Type 1 Diabetes (T1D). Discovery of disease-related epitopes has focused exclusively on peptides originating from native β-cell proteins. However, translational errors may also represent a major source of antigenic peptides. The inventors of the present invention investigated whether the extraordinarily high translation rate of PPI in β-cells combined with β-cell stress during an inflammatory insult leads to generation of defective polypeptides thus enhancing β-cells vulnerability to T-cell mediated destruction. Metabolic or inflammatory stress at the vicinity of the β-cells during insulitis may contribute to the production of neo-antigens to which central tolerance is lacking, triggering autoimmunity. Indeed, the β-cell transcriptome showed increase splicing events under pathophysiological conditions pointing to neo-antigen generation during insulitis (Eizirik et al. The human pancreatic islet transcriptome: expression of candidate genes for type 1 diabetes and the impact of proinflammatory cytokines. PLoS Genet 8:e1002552, 2012; De Jong et al. Post-transcriptional control of candidate risk genes for type 1 diabetes by rare genetic variants. Genes Immun 14:58-61, 2013). In addition, it appears conceivable that the proteome of stressed β-cells expands by post-translational modification processes. The recent discovery of T-cell autoreactivity against deaminated autoantigens underscores the increasing heterogeneity of autoantigenic proteins generated by inflammed β-cells (Van Lummel et al. Posttranslational modification of HLA-DQ binding islet autoantigens in type 1 diabetes. Diabetes 63:237-247, 2014; Van Lummel et al. Changing faces, unmasking the beta-cell: post-translational modification of antigens in type 1 diabetes. Curr Opin Endocrinol Diabetes Obes 20:299-306, 2013). When stressed, the cellular equilibrium in β-cells might be perturbed, affecting high fidelity transcriptional and translational processes during conversion of the genetic information into proteins. Uncontrolled cell proliferation correlates also in tumors with enhanced translation rates and accumulation of aberrant translation products.

In the pancreas, β-cells are insulin factories entirely dedicated to the maintenance of glucose homeostasis. Insulin represents 10-15% of the protein content of a β-cell and it is stored in secretory granules. Upon glucose challenge, insulin is released into the circulation by exocytosis and insulin mRNA is rapidly translated by polysomes to increase insulin biosynthesis. Paradoxically, native insulin and its precursors is also known as the primary autoantigen in T1D. Fragments of the signal peptide of PPI were uncovered as a major source of antigen class I derived epitope targeted by cytotoxic islet autoreactive CD8 T-cells. The inventors of the present invention further investigated whether dysfunctional β-cells produce aberrant translation products (such as DRiP and Splice variants) and hence, whether the extraordinarily high insulin-translation rate leads to generation of non-conventional diabetogenic epitopes.

Polypeptides Comprising Epitopes

The present invention relates to a Defective Ribosomal Product (DRiP) from the human PPI mRNA identified for the first time by the present inventors (also referred to as INS-DRiP herein). DRiPs arise from translation of untranslated regions (UTRs), ribosomal frame-shifting or alternative translation initiation and several have been identified in the art (Berglund et al. Viral alteration of cellular translational machinery increases defective ribosomal products. J Virol 81:7220-7229, 2007; Qian et al. Characterization of rapidly degraded polypeptides in mammalian cells reveals a novel layer of nascent protein quality control. J Biol Chem 281:392-400, 2006; Yewdell. Amsterdamming DRiPs. Mol Immunol 55:110-112, 2013). Alternative splicing of pre-messenger RNAs is a ubiquitous and flexible mechanism for the control of gene expression in eukaryotic cells. It provides cells with the opportunity to create a diversity of protein isoforms. A DRiP polypeptide expressed from the human PPI mRNA according to the invention is referred to as INS-DRiP hereinafter.

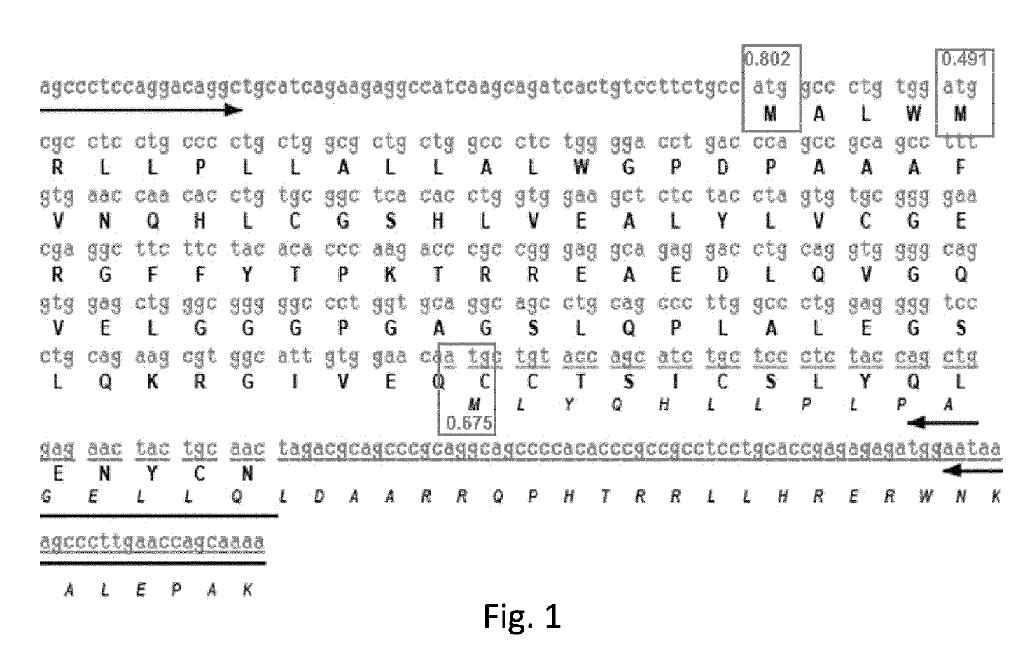

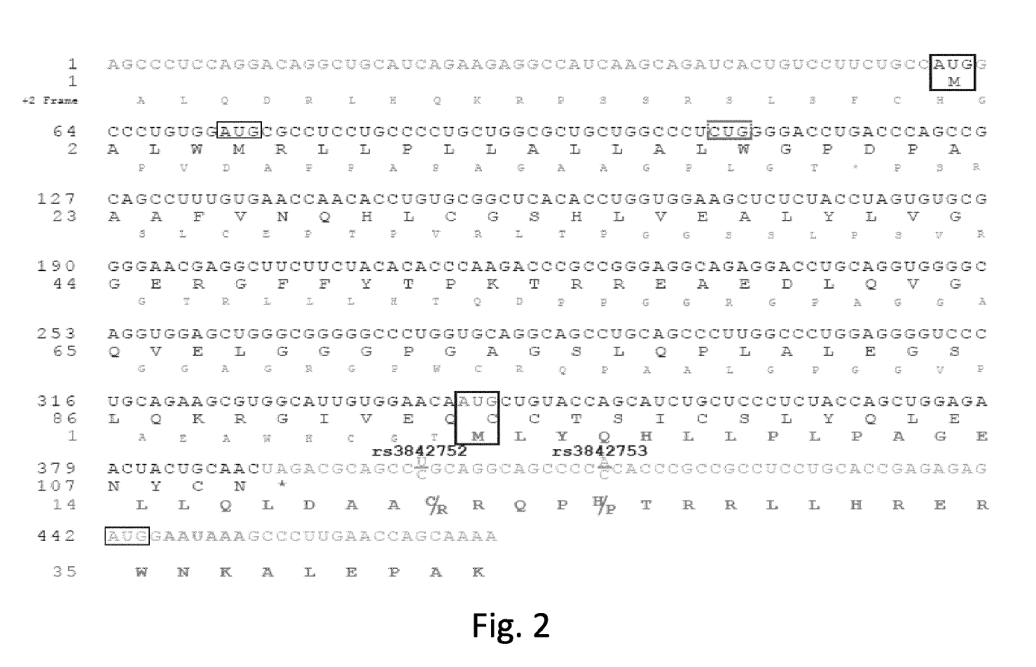

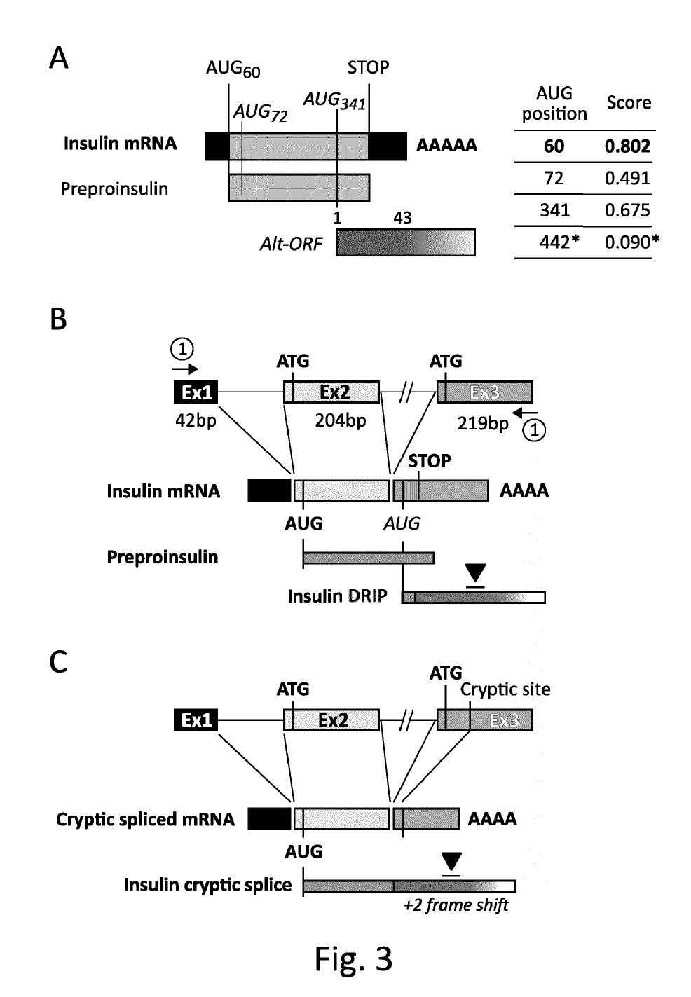

As outlined in more details in Example 1 and FIGS. 1-3, this novel DRiP is translated from an out-of-frame AUG start codon within said human PPI mRNA. The start codon of the here identified DRiP is AUG341 and the open reading frame (ORF) extends into the 3′-UTR region of PPI mRNA and gives rise to a 43 amino acid long polypeptide (see FIG. 2). The 3′-UTR region of PPI mRNA comprises two SNPs, rs3842752 and rs3842753. These so-called pleiotropic SNPs, in particular the RP SNPs, have been described to be associated with increased risk for development of T1D (Marchand et al., Diabetes, 2007; Onengut et al., Nature Genetics, 2015). While these SNPs do not affect insulin sequence and have been discarded as functionally relevant because of their location within the INS 3′UTR3, they are part of the coding region of the INS-DRiP variants according to the present invention, and gives rise to amino acids cysteine (C) or arginine (R) on position 21 and amino acids histidine (H) or proline (P) on position 26 of the INS-DRiP polypeptide, where the position refers to the number of amino acids as counted from the N-terminal of the INS-DRiP polypeptide. Accordingly the ORF of INS-DRiP gives rise to four different isoforms of the INS-DRiP polypeptide, namely the isoforms wherein the first SNP encodes a cysteine (C) and the second SNP encodes either a histidine (H) or a proline (P) and the isoforms wherein the first SNP encodes a an arginine (R) and and the second SNP encodes either a histidine (H) or a proline (P). The four isoforms are herein referred to as INS-DRiP-CH, INS-DRiP-CP, INS-DRiP-RH and INS-DRiP-RP, respectively, and are all objects of the present invention. The inventors of the present invention have found that the INS-DRiP polypeptides according to the present invention are mainly translated as the two isoforms containing the two single SNPs CH and RP, respectively, i.e. INS-DRiP-CH and INS-DRiP-RP. In addition to these DRiPs, a splice variant of the human insulin gene was identified, herein referred to as Splice-RP or Splice-PPI, which terms are used interchangeably. This splice variant is translated as one isoform (RP) partially sharing sequence identity with the genuine insulin gene translation product.

The amino acid sequences of INS-DRiP-CH, INS-DRiP-RP and Splice-RP are as follows, with the SNPs marked in bold:

INS-DRIP-CH:
(SEQ ID NO: 3)
MLYQHLLPLPAGELLQLDAACRQPHTRRLLHRERWNKALEPAK
INS-DRIP-RP:
(SEQ ID NO: 4)
MLYQHLLPLPAGELLQLDAARRQPPTRRLLHRERWNKALEPAK
Splice-RP:
(SEQ ID NO: 5)
MALWMRLLPLLALLALWGPDPAAAFVNQHLCGSHLVEALYLVCGERGFFY
TPKTRREAEDLQAGELLQLDAARRQPPTRRLLHRERWNKALEPAK

The sequence of the INS-DRiP peptide identified as target for CTLs and identified as being naturally processed and presented by T1D high-risk HLA-DQ8cis/trans molecules on DCs (MLYQHLLPL) is underlined in INS-DRiP-CH and INS-DRiP-RP. This peptide is also referred as INS-DRiP₁₋₉ herein and has the amino acid sequence provided as SEQ ID NO:8.

The sequence of the Splice-RP peptide identified as being naturally processed and presented by T1D high-risk HLA-DQ8cis/trans molecules on DCs is underlined in the Splice-RP sequence. This T-cell epitope has the amino acid sequence TRREAEDLQAGELLQLDA (SEQ ID NO:6). The sequence that is homologous with the genuine insulin gene translation product is shown in italic in Splice-RP. The Splice-RP polypeptide is also translated with the same two single nucleotide polymorphisms as the DRiP-RP variant (bold).

The aberrant INS-DRiP variants do not share sequence identity with the genuine (wild type; original) insulin gene translation product (PPI) and the splice variant only partially shares sequence identity with PPI.

These novel INS-DRiP polypeptides, nucleic acids encoding said INS-DRiP polypeptides and nucleic acids complementary (anti-sense) to the nucleic acids encoding said INS-DRiP polypeptides are all objects of the present invention. In particular, in one embodiment of the present invention, the present invention relates to INS-DRiP polypeptides comprising or having the amino acid sequence of SEQ ID NO:3 or SEQ ID NO:4.

In one embodiment, the present invention relates to a polypeptide or a peptide comprising an epitope present in the novel Defective Ribosomal Product (DRiP) from the human PPI mRNA disclosed herein.

Epitopes derived from exogenous islet-autoantigens are presented to CD4+ T cells after a sequence of events termed natural processing and presentation (Blum J S et al. 2013. Pathways of antigen processing. Annu Rev Immunol 31:443-473). Exogenous antigens are taken up by professional antigen-presenting cells (APCs) and loaded in the early endosome where HLA class II molecules reside. Within the late endosome and the lysosome (also in the autophagosome fused with the late endosome and the lysosome), antigens are cleaved by cathepsins and antigenic-peptides are loaded into the HLA class II molecules in the late endosome and transported to the cell membrane where the HLA-loaded natural processed peptides can be presented to CD4+ T cells. Professional APCs, like dendritic cells (DCs) initiate and shape both innate and adaptive immune responses (Banchereau et al. 1998. Dendritic cells and the control of immunity. Nature, 39:245-252). There is increasing know-how about the role of DCs in the pathogenesis of T1D from clinical studies and experimental models (Park J et al. 2013. Chemokine programming dendritic cell antigen response: part I—select chemokine programming of antigen uptake even after maturation. Immunology 139:72-87; Ganguly D et al. 2013. The role of dendritic cells in autoimmunity. Nat Rev Immunol 13:566-577; Turley S et al. 2003. Physiological beta cell death triggers priming of self-reactive T cells by dendritic cells in a type-1 diabetes model. J Exp Med 198:1527-1537). For example, DCs in the pancreatic islets constitutively present beta-cell antigen derived peptides in HLA class II molecules (Calderon B et al. 2008. Dendritic cells in islets of Langerhans constitutively present beta cell-derived peptides bound to their class II MHC molecules. Proc Natl Acad Sci USA 105:6121-6126) which is promoted by inflammatory cytokines (Diana J et al. 2013. Crosstalk between neutrophils, B-1a cells and plasmacytoid dendritic cells initiates autoimmune diabetes. Nat Med 19:65-73). In addition, a recent study highlights the limited role for B-cells as APCs; it was shown that restricting MHC class II expression to B-cells constrained secondary CD4 T cell responses (Archambault A S et al. 2013. Cutting edge: Conditional MHC class II expression reveals a limited role for B cell antigen presentation in primary and secondary CD4 T cell responses. J Immunol 191:545-550). DCs pulsed with islet proteins (PPI, IA-2, GAD65) naturally process islet protein derived peptides presented by T1D high-risk HLA-DQ8cis/trans molecules that are genuine targets for autoreactive CD4 T cells in T1D patients (van Lummel, Diabetes, 2015). The inventors of the present invention investigated which naturally processed peptides derived from the aberrant INS-DRiP variants and the Splice-RP variant are presented by T1D high-risk HLA-DQ molecules expressed on DCs. Two peptides were newly identified to be presented by HLA-DQ8cis/trans molecules: one peptide that is completely homologous to the HLA-A2 DRiP peptide disclosed herein, and a second peptide derived from the Splice-RP variant, which partially overlaps with the genuine proinsulin sequence (FIGS. 1 and 2).

The inventors of the present invention are the first to identify a CD4 and CD8 T cell epitope from a DRiP, in this case the aberrant PPI referred to as INS-DRiP herein. The epitope from the INS-DRiPs identified with the methods disclosed herein contained the amino acid sequence MLYQHLLPL (SEQ ID NO: 8), which is located in the INS-DRiP at position 1 to 9 and referred to as INS-DRiP₁₋₉ herein. This epitope is presented by both T1D high-risk HLA-A2 as well as HLA-DQ8cis/trans molecules. The identification of this novel peptide will add in the understanding of the mechanisms of beta cell stress providing an opportunity to develop therapeutics preventing β-cell stress and T1D pathogenesis that is a result of such β-cell stress. The present invention therefore also relates to methods to identify INS-DRiP-derived peptides, to the INS-DRiP-derived peptides themselves and the use thereof in the diagnosis, prevention and/or treatment of T1D. The inventors of the present invention are also the first to identify a peptide from the aberrant insulin Splice RP product. The peptide with the amino acid sequence TRREAEDLQAGELLQLDA (SEQ ID NO:6) is located in the Splice-PPI at position 54-73.

This means that the present invention also relates to immunogenic fragments of the INS-DRiP and Splice-RP polypeptides which comprise an epitope. Since epitopes can be as short as 5 amino acids, the present invention also relates to peptides comprising down to e.g. 5 amino acids. What is important is that the polypeptide or peptide according to the invention comprises an epitope capable of eliciting a specific immunological binding either by itself or as part of and MHC complex, where the MHC complex is preferably HLA. Preferably, the HLA forming part of a complex with a polypeptide or peptide according to the invention is selected from the group consisting of HLA-A2, and/or HLA-DQ8cis, more preferably HLA-DQ2/8 and even more preferably by T1D highest-risk HLA-DQ8trans, expressed in DQ2/8 heterozygous individuals.

Accordingly, polypeptides or peptides according to the present invention can comprise as little as at least 5 consecutive amino acids, such as at least 6, at least 7, at least 8 or at least 9 consecutive amino acids of the amino acids present in INS-DRiP and/or Splice-RP in so far as these consecutive amino acids constitute an epitope. As used herein, the term “polypeptide” is intended to mean an amino acid sequence of at least 10 amino acids, such as e.g. from 10 to 100 amino acids. As used herein, the term “peptide” is intended to mean an amino acid sequence of less than 10 amino acids, such as e.g. from 5 to 10 amino acids.

Examples of suitable epitopes according to the present invention are epitopes comprising an amino acid sequence selected from the group consisting of TRREAEDLQAGELLQLDA (SEQ ID NO:6), LHRERWNKALEPAK (SEQ ID NO: 7), MLYQHLLPL (SEQ ID NO: 8) and PTRRLLHRE (SEQ ID NO: 9). Preferably, a peptide according to the invention, is selected from the group consisting of TRREAEDLQAGELLQLDA (SEQ ID NO:6), LHRERWNKALEPAK (SEQ ID NO: 7), MLYQHLLPL (SEQ ID NO: 8) and PTRRLLHRE (SEQ ID NO: 9).

The amino acid sequence TRREAEDLQAGELLQLDA (SEQ ID NO:6) is located in the Splice-PPI at position 54-73 and is unique for the Splice-RP polypeptide.

The amino acid sequence LHRERWNKALEPAK (SEQ ID NO:7) is located in the very C-terminal of INS-DRiP polypeptides and the Splice-RP polypeptide, more precisely at amino acid position 30-43 and in the Splice-PPI at position 82-95. This peptide has been used herein to raise an antiserum for identification of INS-DRiP and/or Splice-RP variants, which antiserum has been used in both western blots and in imaging. LHRERWNKALEPAK (SEQ ID NO:7), located in the INS-DRiP at amino acid position 30-43 and in the Splice-RP at position 82-95.

The amino acid sequence MLYQHLLPL (SEQ ID NO: 8) is located in the very N-terminal of INS-DRiP polypeptides more precisely at amino acid position 1-9. This epitope is also referred to as INS-DRiP₁₋₉. The INS-DRiP₁₋₉ peptide has been identified herein as target for CTLs and as being naturally processed and presented by T1D high-risk HLA-DQ8cis/trans molecules on DCs.

The amino acid sequence PTRRLLHRE (SEQ ID NO: 9) is located in the Splice-PPI at position 77-85 and is unique for Splice-PPI. By employing similar in silico studies, this 9-amino acid potential high-affinity HLA-DQ8cis/trans binding peptide was identified for the Splice RP variant.

In one embodiment of the present invention, a suitable epitope is capable of specifically binding to an antibody. An example of such an epitope is an epitope comprising an amino acid sequence LHRERWNKALEPAK (SEQ ID NO:7).

In another embodiment of the present invention, a suitable epitope is a T-cell epitope. According to the present invention the T-cell epitope suitably comprises the amino acid sequence MLYQHLLPL (SEQ ID NO: 8) or PTRRLLHRE (SEQ ID NO: 9).

In order for the non-conventional polypeptides to be immunogenic (and to play a role in T1D) it needs to generate peptides that efficiently compete with other peptides for presentation on the cell surface by HLA class I and/or class II molecules.

In a preferred embodiment, suitable epitopes according to the present invention are epitopes which are unique for INS-DRiP and which show binding to HLA, preferably HLA-A2 and/or HLA-DQ8cis/trans, more preferably HLA-DQ2/8 and even more preferably by T1D highest-risk HLA-DQ8trans, expressed in DQ2/8 heterozygous individuals. The inventors of the present invention have identified that the INS-DRiP polypeptides according to the invention carry a high affinity HLA-A2 and HLA-DQ8cis/trans binding epitope. This epitope is INS-DRiP₁₋₉ (SEQ ID NO: 8) and accordingly, in a highly preferred embodiment of the invention, the epitope is INS-DRiP₁₋₉(SEQ ID NO: 8).

The present invention also relates to isolated nucleic acids encoding the polypeptides and peptides according to the invention and the nucleic acids complementary thereof. The coding nucleic acids can be used in cloning vectors for expression of the polypeptides or peptides according to the invention.

The polypeptides and peptides according to the invention can be prepared by conventional methods known to the person skilled in the art. Several methods are available in the art for the generation of recombinant and synthetic peptides that can be generated to a pharmaceutically acceptable grade, and used accordingly in methods of treatment.

In one embodiment of the present invention, the polypeptides peptides and nucleic acid sequences are isolated. As used herein, the term “isolated” means that the polypeptide, peptide or nucleic acid according to the invention has been removed from its natural environment. For instance, an “isolated” polypeptide is a polypeptide that has been removed from the cytoplasm or from the outer membrane of a cell, and most of the polypeptides, nucleic acids, and other cellular material of its natural environment are no longer present. An “isolated” polypeptide also includes a polypeptide or peptide produced using recombinant techniques, or chemically or enzymatically synthesized. In yet another embodiment, “Isolated” as used herein means that the polypeptides or peptides are generated such that no contaminants are present and are preferably not cut out a natural protein or from a natural source, but rather that it is produced in a controlled environment for the generation of synthetic peptides, preferably in a sterile environment. It is preferred to produce the polypeptides and peptides of the present invention in a synthetic manner, because it can be well-controlled and no human or cell-derived contaminations are present. Besides that, the production of synthetic peptides is relatively cheap. Accordingly, in a preferred embodiment, the polypeptides and peptides of the present invention are produced in vitro.

The present invention also relates to (isolated) nucleic acid molecules encoding the polypeptides or peptides according to the present invention, and to vectors comprising a nucleic acid according to the invention. The invention further relates to cells transformed with nucleic acids of the present invention. In yet another preferred embodiment, the invention relates to a cell transfected with a nucleic acid encoding any of the polypeptides or peptides according to the present invention, and a MHC class I or II molecule.

The present invention also relates to polypeptides and peptides according to the invention for use in the diagnosis of type 1 diabetes mellitus (also referred to herein as TD1). Furthermore, the present invention relates to use of polypeptides, peptides according to the invention as a biomarker for type 1 diabetes mellitus and/or in detecting β-cell stress.

Cytotoxic T-Cells

Aberrant polypeptides are rapidly detected and degraded by the cellular quality control machinery and represent the major source of HLA class I and II epitopes. Here, the first evidence is presented of such non-conventional epitopes implicated in an autoimmune disease leading to the destruction of human β-cells by cytotoxic CD8 T-cells. The low expression of insulin mRNA detected in the thymus makes the participation of translational mistakes to T-cell education unlikely. Consequently, these error-specific CTLs are part of the normal T-cell repertoire, as reflected by the presence of naïve DRiP-specific CTLs in healthy individuals. In contrast, the higher frequency and the pro-effector phenotype of these CTLs detected in T1D patient PBMC supports a peripheral activation and a role in the immunopathogenesis of T1D.

When polypeptides or peptides according to the present invention comprise a T-cell epitope such epitope may be targeted by T-cells selected from the group consisting of cytotoxic T-cells (CTLs), CD8+ cells, effector CD4 T-cells and regulatory CD4 T-cells. As used herein, a “T-cell targeting a peptide” is intended to mean that the T-cell can recognize and respond to said peptide by its T-cell receptor specifically recognizing and binding to the peptide-MHC complex, such as the peptide-HLA complex. The peptide-HLA complex is expressed on the surface of antigen presenting cells or, as in the case of TD1 patients, on the surface of β-cells. A “T-cell targeting a peptide” is also sometimes referred to as a “T-cell directed against a peptide”. Accordingly, CTLs targeting a polypeptide or peptide according to the invention are also sometimes referred to as “peptide-directed” or “peptide-targeting” CTLs. Likewise, CD8+ T-cells targeting a polypeptide or peptide according to the invention are also sometimes referred to as “peptide-directed” or “peptide-targeting” CD8+ T-cells. When the peptide is INS-DRiP₁₋₉, such cells can also be referred to as “INS-DRiP₁₋₉-directed” or “INS-DRiP₁₋₉-targeted” CTLs or CD8+ T-cells, respectively.

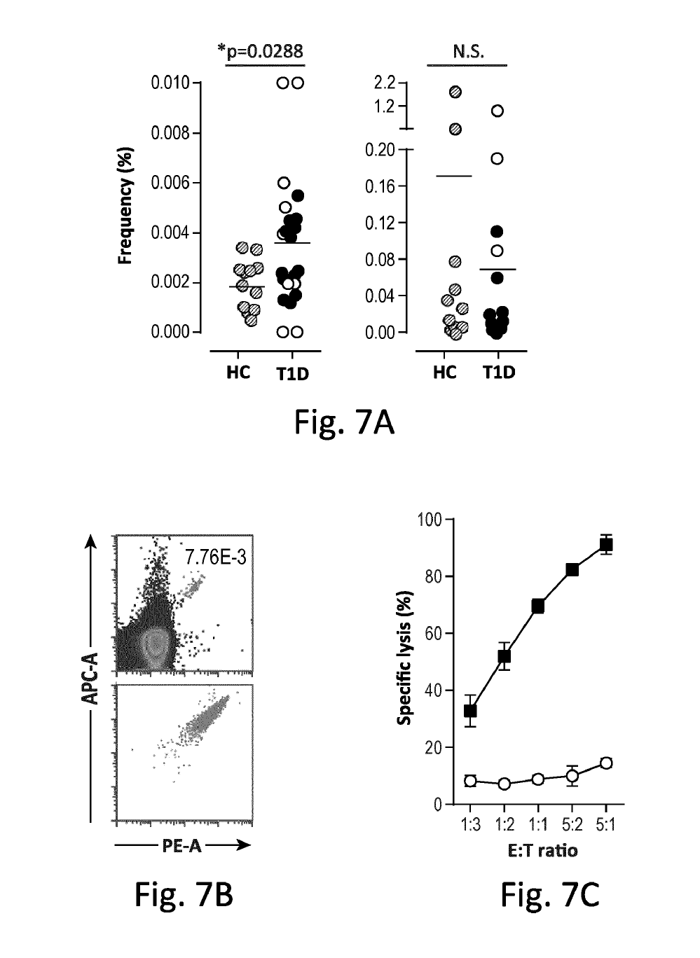

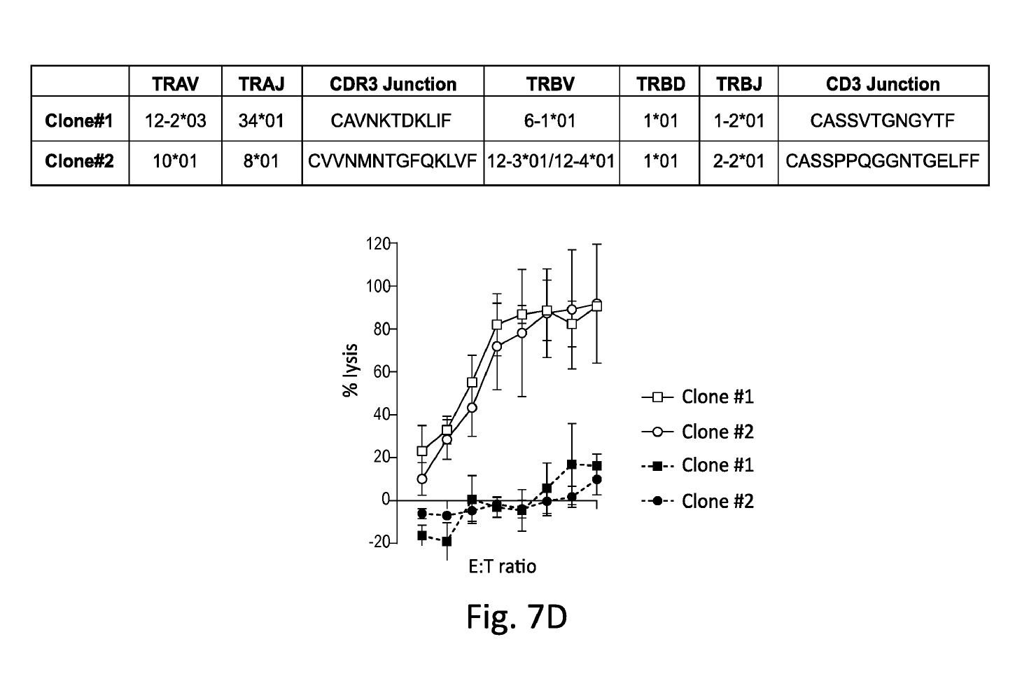

The present inventors have found that CD8+ T-cells directed against the N-terminal peptide of the non-conventional INS-DRiPs are present in the circulation of patients diagnosed with T1D and hence provided direct evidence that such CD8+ T-cells are diabetogenic by specifically killing human β-cells (FIGS. 7C and 9). The study explained in more detail in the accompanying examples appears to be the first demonstrating the participation of CD8 and CD4 epitopes derived from non-native islet proteins in T1D pathology and reveals an unexplored source of non-conventional β-cell polypeptides that act as source of self-epitopes in autoimmune disease. The findings of the present inventors support that CTLs targeting a polypeptide or peptide according to the present invention can be used to detect whether or not a subject, such as a human subject suffers from, or is a risk of suffering from type 1 diabetes mellitus.

The present invention relates to CTLs, such as CD8+ T-cells, targeting a polypeptide or a peptide according to the invention for use in the diagnosis of of type 1 diabetes mellitus. In a highly preferred embodiment, the present invention relates to CTLs, such as CD8+ T-cells, targeting INS-DRiP₁₋₉ for use in the diagnosis of of type 1 diabetes mellitus.

The present invention furthermore relates to use of a cytotoxic T-cell, such as a CD8+ T-cell, targeting said polypeptide or peptide, as a biomarker for type 1 diabetes mellitus and/or in detecting β-cell stress. In a preferred embodiment, the present invention relates to use of CTLs, targeting INS-DRiP₁₋₉ as a biomarker for type 1 diabetes mellitus and/or in detecting β-cell stress. In a highly preferred embodiment, the present invention relates to use of CD8+ T-cells targeting INS-DRiP₁₋₉ as a biomarker for type 1 diabetes mellitus and/or in detecting β-cell stress.

Furthermore, the present invention relates to a method for identifying a subject, such as a human subject, suffering from, or being at risk of suffering from, type 1 diabetes mellitus, the method comprising:
a) measuring the amount of cytotoxic T-cells targeting a polypeptide or peptide according to the invention in a blood sample from said subject; and
b) comparing the measured amount of said cytotoxic T-cells to a reference value, wherein a significant deviation in the amount of measured cytotoxic T-cells compared to the reference value, is indicative of type 1 diabetes mellitus.

Preferably, the blood sample used in this method is a heparinized blood sample. In one embodiment, the measured cytotoxic T-cells are CD8+ T-cells targeting a polypeptide or peptide according to the invention. In a preferred embodiment of this method, the measured cytotoxic T-cells are CD8+ T-cells targeting INS-DRiP₁₋₉.

In yet a further embodiment, step b) of this method comprises comparing the measured amount of cytotoxic T-cells targeting a polypeptide or peptide according to the invention to a reference value reflecting the amount of cytotoxic T-cells targeting a polypeptide or peptide according to the invention in blood samples of a group of subjects not suffering from type 1 diabetes mellitus, wherein an increase in the amount of measured cytotoxic T-cells targeting a polypeptide or peptide according to the invention compared to the reference value, is indicative of type 1 diabetes mellitus. In a preferred embodiment, the subject is a human subject. The type of cytotoxic T-cells measured in the blood sample of the subject and those reflected by the reference value, is the same type of cytotoxic T-cells targeting the same polypeptide or peptide.

The present invention also relates to a method for measuring the amount of cytotoxic T-cells targeting a polypeptide or peptide according to the invention in a sample, such as a blood sample from a subject, wherein the subject is preferably a human subject and the blood sample is preferably a heparinized blood sample. This method may further comprise comparing the measured amount of said cytotoxic T-cells to a reference value. In one embodiment of this method, the measured cytotoxic T-cells are CD8+ T-cells targeting a polypeptide or peptide according to the invention. In a further embodiment the measured cytotoxic T-cells, such as measured CD8+ T-cells, are targeting INS-DRiP₁₋₉. In a preferred embodiment of this method, the measured cytotoxic T-cells are CD8+ T-cells targeting INS-DRiP₁₋₉. The type of cytotoxic T-cells measured in the blood sample of the subject and those reflected by the reference value, is the same type of cytotoxic T-cells targeting the same polypeptide or peptide.

Several methods for measuring the amount of T-cells targeting a specific peptide are known to a person skilled in the art and can be adjusted to the above-mentioned methods by using means for specifically binding and/or labelling the cytotoxic T-cells, such as measured CD8+ T-cells, targeting a peptide of the present invention thereby allowing said cells to be measured. Such means can for example be isolated peptide-MHC complexes according to the invention, or multimers thereof, such as tetramers or multimers thereof. These peptide-MHC complexes can suitably be connected to nanoparticles, such as, quantum dots (Qdot), allowing for detection e.g. by FACS.

Peptide-MHC Complex

The invention also relates to an isolated peptide-MHC complex, wherein the peptide forming part of the complex is a polypeptide or peptide according to the present invention. The MHC part of the peptide-MHC complex is a an MHC class I or II and preferably a HLA. More preferably, the HLA forming part of a complex with a polypeptide or peptide according to the invention is selected from the group consisting of HLA-A2, and/or HLA-DQ8cis/trans, more preferably HLA-DQ2/8 and even more preferably by T1D highest-risk HLA-DQ8trans, expressed in DQ2/8 heterozygous individuals. Preferably the isolated peptide-MHC complex according to the invention is soluble. In a highly preferred embodiment, the peptide forming part of the peptide-MHC is INS-DRiP₁₋₉ (SEQ ID NO: 8). Methods for preparing an isolated peptide-MHC complex are known to a person skilled in the art and is for instance described in Abreu et al. CD8 T cell autoreactivity to preproinsulin epitopes with very low human leukocyte antigen class I binding affinity. Clin Exp Immunol 170:57-65, 2012, which is incorporated herein by reference.

The present invention also relates to use of a peptide-MHC complex according to the invention for applications requiring means of specifically binding a CTL, CD8+ T-cell or a TCR. Examples of such applications are isolation, in vivo and in vitro imaging, drug targeting and measuring the amount (counting) such cells, but the invention is not limited to these examples. In a preferred embodiment of the present invention the peptide-MHC according to the present invention is used in the methods for measuring the amount of peptide-directed cytotoxic T-cells according to the invention.

Binding Molecules

The present invention also relates to an isolated binding molecule that has binding affinity for or is capable of specifically binding a polypeptide or peptide according to the present invention. The present invention also relates to an isolated binding molecule that has binding affinity for or is capable of specifically binding a peptide-MHC complex according to the invention.

In one embodiment, such isolated binding molecule is an antibody. Antibodies may come in a wide variety of forms, e.g. either humanized or as hybrids, or fragments; all well-known to the person skilled in the art. Accordingly, antibodies according to the present invention can suitably be selected from the group consisting of a chimeric antibody, a human antibody, a humanized antibody, a single chain antibody, a bispecific antibody, a Fab fragment, a nanobody and a non-fucosylated antibody. Furthermore, the present invention also relates to functional fragments of said antibodies, such as, functional therapeutic/diagnostic relevant fragments thereof. The invention also relates to an antibody or functional fragment thereof, or a polyclonal antiserum, raised and/or directed against a polypeptide or peptide according to the present invention, such as, an INS-DRiP of SEQ ID NO:3 or 4, or an immunogenic part thereof; or raised and/or directed against a splice variant of PPI, wherein said splice variant comprises the amino acid sequence of SEQ ID NO:5, or an immunogenic part thereof. The polyclonal antibodies according to this embodiment of the present invention is suitably in the form of a polyclonal antiserum raised and/or directed against a polypeptide or peptide according to the invention. Preferably, said polyclonal antiserum is raised and/or directed against the peptide of LHRERWNKALEPAK (SEQ ID NO:7) or MLYQHLLPL (SEQ ID NO: 8), or an immunogenic fragment thereof.

An isolated binding molecule according to the present invention, such as e.g. an antibody according to the present invention, can suitably be monoclonal or polyclonal. Methods for the preparation of an isolated binding molecule, according to the invention, such as an antibody, are known to a person skilled in the art. In particular methods for the preparation of the above-mentioned different types of antibodies and antiserums are considered routine to the person skilled in the art.

In another embodiment this isolated binding molecule comprises complementarity-determining regions (CDRs) derived from a TCR capable of specifically binding a peptide-MHC complex according to the invention, for example an isolated antibody comprising CDRs derived from a TCR capable of specifically binding a peptide-MHC according to the invention. The invention also relates to a T cell receptor (TCRs) that has specific binding affinity for a peptide-MHC complex according to the present invention, preferably comprising both a TCR alpha chain variable domain and a TCR beta chain variable domain. Such TCR is preferably soluble. An isolated binding molecule according to the present invention, which is capable of specifically binding a peptide-MHC complex according to the present invention, such as e.g. a TCR, a soluble TCR or an antibody derived from a TCR and comprising CDRs of a TCR, can suitably be monoclonal or polyclonal. Methods for the preparation of an isolated binding molecule capable of specifically binding a peptide-MHC complex according to the invention are known to a person skilled in the art.

The inventors of the present invention also generated a polyclonal antiserum directed against the C-terminal part of INS-DRiP and Splice-RP. It was shown that this polyclonal serum was very useful in immunohistochemistry. Expression of the aberrant insulin gene products (INS-DRiP and/or Splice-RP) in human islets displays a “patchy” vitiligo-like histology as studied by immunohistochemistry (data not shown). This provides the first direct proof of the heterogeneous disease course related to aberrant islet (neo)antigens. The antiserum was raised against a partial sequence of the splice variant, namely LHRERWNKALEPAK (SEQ ID NO:7), located in the INS-DRiP at amino acid position 30-43 and in the Splice-RP at position 82-95. The polyclonal antibodies comprised in this antiserum are also referred to as DRiP/Splice polyclonal antibodies (Poab).

The present invention also relates to an isolated binding molecule according to the invention or a polyclonal antiserum according to the invention for use in the diagnosis of type 1 diabetes mellitus.

Furthermore, the present invention relates to use of an isolated binding molecule according to the invention or a polyclonal antiserum according to the invention in in vivo and in vitro imaging, for drug targeting, in the diagnosis of type 1 diabetes mellitus or in detecting β-cell stress. Hence, in a further aspect, the invention relates to methods of imaging, and preferably the staining of β-cells, using the antibodies or antisera of the present invention.

Furthermore, the present invention relates to a method for identifying a subject, such as a human subject, suffering from, or being at risk of suffering from, type 1 diabetes mellitus, the method comprising:
a) measuring the amount of polypeptide or peptide according to the invention in a blood, serum or plasma sample from said subject; and
b) comparing the measured amount of said polypeptide or peptide to a reference value,

wherein a significant deviation in the amount of measured polypeptide or peptide compared to the reference value, is indicative of type 1 diabetes mellitus.

Preferably, the sample used in this method is a serum or a plasma sample. In one embodiment, step b) of this method comprises comparing the measured amount of polypeptide or peptide according to the invention to a reference value reflecting the amount of corresponding polypeptide or peptide according to the invention in corresponding blood samples of a group of subjects not suffering from type 1 diabetes mellitus, wherein an increase in the amount of measured polypeptide or peptide compared to the reference value, is indicative of type 1 diabetes mellitus. In a preferred embodiment, the subject is a human subject. In a highly preferred embodiment, the measured peptide according to the invention is INS-DRiP₁₋₉.

The present invention also relates to a method for measuring the amount of a polypeptide or peptide according to the invention in a sample, such as a blood, serum or plasma sample from a subject, wherein the subject is preferably a human subject and the blood sample is preferably a serum or plasma sample. This method may further comprise comparing the measured amount of said polypeptides or peptides to a reference value. In a preferred embodiment of this method, the measured peptide according to the invention is INS-DRiP₁₋₉.

Several methods for measuring the amount of polypeptides or peptides in biological samples are known in the art and can be adjusted to the above-mentioned methods by using means for specifically binding and/or labelling the polypeptides or peptides according to the present invention. Such means can for example be an isolated binding molecule according to the present invention capable of specifically binding a polypeptide or peptide according to the present invention. In a preferred embodiment the isolated binding molecule according to the invention used for detection, is an antibody according to the invention. Antibodies according to the invention can for instance be linked to an enzyme or a fluorescent group allowing for quantification of the polypeptide or peptide e.g. by ELISA or RIA, respectively.

Peptide Immunotherapy and Tolerance Promoting Cells

The present invention also relates to use of a polypeptide or a peptide according to the present invention and representing the an epitope within the DRiP and/or Splice variants in peptide immunotherapy by which CD4+ T cells that are involved in T1D are inactivated, through which long-term β-cell tolerance can be restored. The polypeptide or peptide of the present invention may be used in a purified manner, synthesized to GMP grade, and applied according to methods known in the art. The peptide may be given via intradermal, subcutaneous or intravenous injection, or via other parenteral, oral or topical routes. In a preferred embodiment, the peptide of the present invention is used in conjunction with tolerance promoting cells (also referred to herein as tolerogenic cells). Especially preferred cells that are used for this purpose are immature dendritic cells and dendritic cells, preferably treated with vitamin D3 or its analogues. When immature dendritic cells are used, such cells are obtained from the patient's blood, expanded in vitro using standard techniques, followed by binding of the peptide(s) to the cells (called peptide pulsing). Peptide-loaded cells are re-introduced into the patient via any of the parenteral routes known to the person skilled in the art. Treatment following this protocol may be continued for several weeks, months or years according to the primary outcome measures, such as a change in an increase of peptide-induced IL-10+ peptide-reactive cells or a decrease in IFN-γ+ peptide-reactive cells. Any lower requirement of external insulin dosing upon and during treatment, will be indicative of enhancement of endogenous insulin production. The presence or re-appearance of for example IFN-γ+ cells recognizing the therapeutic peptide of the present invention will dictate continuation of the therapy. The patients that are treated with the peptide of the present invention, or with the dendritic cells presenting said peptide will preferably have at least one high risk HLA molecule such as HLA-DR3, HLA-DR4, HLA-DQ2 or HLA-DQ8, as a homozygote, but more preferably as a heterozygote, such as the highest risk HLA-DQ8trans molecule.

The invention also relates to a culture of tolerance promoting cells targeting a polypeptide or peptide according to the present invention. The tolerance promoting cells are suitably selected from the group consisting of immature dendritic cells and dendritic cells.

Furthermore, the present invention relates to a composition comprising a polypeptide or a peptide according to the invention. Such a composition can be used in peptide immunotherapy applications using the naturally processed and presented polypeptides or peptides according to the present invention. A preferred peptide for peptide immunotherapy of type 1 diabetes mellitus is the INS-DRiP₁₋₉, which is shown by the present inventors to be naturally processed and presented. In a preferred embodiment, a composition according to the invention therefore comprises the INS-DRiP₁₋₉ peptide. Advantageously, a composition according to the present invention further comprises a culture of tolerance promoting cells and/or a tolerance promoting adjuvant. The tolerance promoting adjuvant is suitably Vitamin D3 and preferably active vitamin D3 and more preferably 1,2(OH)₂ vitamin D3. Such compositions are suitable for use in the prevention treatment of T1D.

In one embodiment, the composition according to the present invention comprises a peptide according to the present invention and a culture of tolerance promoting cells. Preferably the peptide used is INS-DRiP₁₋₉. The tolerance promoting cells are preferably dendritic cells generated from autologous monocytes that have been modulated in culture to display a tolerogenic phenotype and function (hereinafter referred to as TolDC). The tolerance promoting cells can prepared by isolating monocytes, such as preferably CD14+ cells from a leukapheresis product from a patient. After culturing these cells under suitable conditions to become anti-inflammatory with GM-CSF, IL-4 and active vitamin D3, preferably 1,2(OH)₂ vitamin D3, on day 1 and dexamethasone on day 3, the obtained cells differentiate in 6 days to become anti-inflammatory immature dendritic cells. Suitably, the active Vitamin D3 is added to the cell culture at a concentration of 2×10⁻⁸ M. The obtained cells can suitably be cryopreserved until they are used for treatment. Before treatment, cells are thawed if relevant. Next, the cells are matured in a suitable maturing culture medium. Preferably, the maturing culture medium comprises Granulocyte-macrophage colony-stimulating factor (GM-CSF), TNFα, IL-6, IL-1β and/or PGE2 in suitable amounts. Even more preferably, the maturing culture medium comprises Granulocyte-macrophage colony-stimulating factor (GM-CSF), TNFα, IL-6, IL-1β and PGE2 in suitable amounts. The obtained mature tolerance promoting cells are then loaded with peptide. Peptide loading of tolerance promoting cells is done by contacting the cells with a suitable amount of peptide for a suitable amount of time at a suitable temperature. An example of such suitable conditions is subjecting tolerance promoting cells at a cell concentration of 0.5×10⁶/ml to 4 μM peptide for 4 hrs at 37° C. Preferably the peptide used is INS-DRiP₁₋₉. The obtained composition of peptide-loaded tolerance promoting cells is suitably administered with an interval of from about 20 to about 36 days, such as from about 22 to about 34 days, from about 24 to about 32 days, from about 26 to about 30 days, such as at an interval of about 28 days. Suitable routes of administration are by intradermal, subcutaneous or intravenous injection, or via other parenteral or topical routes. In a preferred embodiment, the composition is administered by intradermal injections. Preferably, the composition is administered in a dosage of between from about 0.5×10⁷ to about 0.5×10⁷ tolerance promoting cells.

The present invention also relates to a composition according to the invention for use as a medicament. Furthermore, the present invention relates to a composition according to the invention for use in a method for the prevention and/or treatment of type 1 diabetes mellitus. Furthermore the present invention relates to a composition according to the present invention, for use in a method for the prevention and/or treatment of type 1 diabetes mellitus, wherein said composition is administered at an interval of from about 20 to about 36 days, such as from about 22 to about 34 days, from about 24 to about 32 days, from about 26 to about 30 days, such as at an interval of about 28 days.

Preferably a composition according to the present invention is a pharmaceutical composition. Such pharmaceutical composition may further comprise a vehicle and/or an excipient, such as a diluent or carrier.

Further Applications

The findings of the present inventors renders isolated polypeptides or peptides according to the invention, the isolated nucleic acids according to the invention, the isolated peptide-MHC complexes according to the invention, the isolated binding molecules according to the invention, the polyclonal antiserums according to the invention, the cultures of tolerance promoting cells according to the invention and the compositions according to the invention broadly applicable for a variety of different purposes, some of which being diagnosis, prevention and treatment of type 1 diabetes mellitus, drug targeting, peptide immunotherapy, detection of β-cell stress, in vivo and in vitro imaging and methods for isolation, purification and/or quantification of molecules and/or cells. For all of these different application areas the underlying mechanism is the specific binding between the involved molecules. These specific bindings renders it possible to isolate, purify and quantify a respective specific binding partner of a given molecule, where the types of specific bindings in play are nucleic acid hybridizations, antibody-epitope interactions and the interaction between a TCR and the peptide-MHC complex or an antibody and the peptide-MHC complex. As used herein, the term “binding partner” means a biologically complementary molecule or complex capable of specifically binding to the molecule or complex in question. According to this definition, complementary nucleic acids are binding partners to one another, an antibody is a binding partner of an epitope and vice versa and a TCR is a binding partner of a peptide-MHC complex and vice versa. Isolation and purification can for instance be performed by immuno-precipitation and quantification can for instance be performed by ELISA and RIA. Drug targeting applications and in vivo and in vitro imaging is also taking advantage of such specific bindings as for instance those between an antibody and its epitope, as will be known to the person skilled in the art. Many other methods are known in the art which takes advantage of such specific interactions, and the skilled person will know how to use these methods employing the molecules and complexes provided by the present invention.

In one embodiment, the present invention relates to use of an isolated polypeptide or peptide according to the invention, an isolated nucleic acid according to the invention, an isolated peptide-MHC complex according to the invention, an isolated binding molecule according to the invention or a polyclonal antiserum according to the invention for drug targeting.

In another embodiment, the present invention relates to use of an isolated polypeptide or peptide according to the invention, an isolated nucleic acid according to the invention, an isolated peptide-MHC complex according to the invention, an isolated binding molecule according to the invention or a polyclonal antiserum according to the invention for isolation, purification and/or quantification of a binding partner or a cell comprising a binding partner.

In another embodiment, the present invention relates to use of an isolated polypeptide or peptide according to the invention, an isolated nucleic acid according to the invention, an isolated peptide-MHC complex according to the invention, an isolated binding molecule according to the invention or a polyclonal antiserum according to the invention in in vitro and in vivo imaging.

In another embodiment, the present invention relates to use of an isolated polypeptide or peptide according to the invention, an isolated nucleic acid according to the invention, an isolated peptide-MHC complex according to the invention, an isolated binding molecule according to the invention or a polyclonal antiserum according to the invention in the diagnosis of type 1 diabetes mellitus.

In another embodiment, the present invention relates to use of an isolated polypeptide or peptide according to the invention, an isolated nucleic acid according to the invention, an isolated peptide-MHC complex according to the invention, an isolated binding molecule according to the invention or a polyclonal antiserum according to the invention in detecting β-cell stress.

In another embodiment, the present invention relates to use of an isolated polypeptide or peptide according to the invention, an isolated nucleic acid according to the invention, an isolated peptide-MHC complex according to the invention, an isolated binding molecule according to the invention, a polyclonal antiserum according to the invention, a culture of tolerance promoting cells according to the invention or a composition according to the invention for the prevention and/or treatment of type 1 diabetes mellitus.

In another embodiment, the present invention relates to an isolated polypeptide or peptide according to the invention, an isolated nucleic acid according to the invention, an isolated peptide-MHC complex according to the invention, an isolated binding molecule according to the invention or a polyclonal antiserum according to the invention for use in the diagnosis of type 1 diabetes mellitus.

In another embodiment, the present invention relates to an isolated polypeptide or peptide according to the invention, an isolated nucleic acid according to the invention, an isolated peptide-MHC complex as according to the invention, an isolated binding molecule according to the invention, a polyclonal antiserum according to the invention, a culture of tolerance promoting cells according to the invention or a composition according to the invention for use as a medicament.

In another embodiment, the present invention relates to use of an isolated polypeptide or peptide according to the invention, an isolated nucleic acid according to the invention, an isolated peptide-MHC complex as according to the invention, an isolated binding molecule according to the invention, a polyclonal antiserum according to the invention, a culture of tolerance promoting cells according to the invention or a composition according to the invention for the manufacture of a medicament.

In another embodiment, the present invention relates to an isolated polypeptide or peptide according to the invention, an isolated nucleic acid according to the invention, an isolated peptide-MHC complex according to the invention, an isolated binding molecule according to the invention, a polyclonal antiserum according to the invention, a culture of tolerance promoting cells according to the invention or a composition according to the invention for use in the prevention and/or treatment of type 1 diabetes mellitus.

In another embodiment, the present invention relates to use of an isolated polypeptide or peptide according to the invention, an isolated nucleic acid according to the invention, an isolated peptide-MHC complex as according to the invention, an isolated binding molecule according to the invention, a polyclonal antiserum according to the invention, a culture of tolerance promoting cells according to the invention or a composition according to the invention for the manufacture of a medicament for the prevention, diagnosis or treatment of type 1 diabetes mellitus.

In yet another embodiment, the present invention relates to a method of treating a human subject having type 1 diabetes mellitus, or that is at risk of developing type 1 diabetes mellitus, said method comprising administering to said human subject an effective amount of an isolated polypeptide or peptide according to the invention, an isolated nucleic acid according to the invention, an isolated peptide-MHC complex according to the invention, an isolated binding molecule according to the invention, a polyclonal antiserum according to the invention, a culture of tolerance promoting cells according to the invention or a composition according to the invention.

The invention also relates to a method of treating T1D comprising administering any of the peptides, nucleic acids encoding any one of said peptides, peptide-MHC complexes, or soluble TCRs according to the present invention, to a subject suffering from T1D.

The present invention provides new peptides, compositions, methods and uses as claimed in the appended claims and as outlined in more detail herein. The invention has sought to solve (at least partially) the problems that exist in the art related to peptides that have been identified thus far and that are held to be useful in the diagnosis, prevention and/or treatment of T1D. The peptides of the present invention may be used in specific medical applications for the treatment or prevention of T1D. In such applications, regulatory populations of T cells (Tr1 cells) are induced that recognize the same peptide as the equivalent effector pathogenic T cells (these cells produce IFN-γ and are called Th1). Peptide immunotherapy is particularly potent at inducing Tr1 cells that produce the immunosuppressive cytokine IL-10. Under these circumstances, when the peptide of the present invention is presented by an APC in the pancreas or local lymph nodes in a patient developing or suffering from T1D, the peptide is recognized simultaneously by Th1 and Tr1 cells. It is known that under these circumstances the Tr1 cell is dominant and exercises ‘bystander suppression’ over the Th1 response.

The data presented herein propose a new plausible explanation for β-cell destruction by the immune system: either i) the insulin exon 2 pre-messenger splicing that would position AUG₃₄₁ as the first AUG encountered by the ribosome during scanning, or ii) ribosome scan-through of the genuine AUG resulting in translation initiation at a downstream AUG on the mature insulin mRNA. mRNA analysis of human pancreatic islets under normal or pathogenic conditions have not revealed such splicing variant (data not shown). The alternative translation initiation seems the most likely explanation. Similar initiation mechanisms have been shown to be involved in the regulation of ATF4 mRNA and ATF5 mRNA translation under stress stimulation. β-cells are highly sensitive to stress. Moreover, environmental stress may differently affect degradation of insulin by-products, and hence increasing INS-DRiP epitope presentation; the absence of an in-frame stop codon in the generation of the INS-DRiP polypeptide implying the participation of distinct elimination mechanisms. Given the extraordinarily high PPI translation rate in β-cells, even rare mistakes in translation can represent a substantial source of neo-antigens. Killing experiments on human pancreatic β-cells prove that the epitope of the present invention is indeed produced, processed and presented. This appears to be the first study that explored non-conventional polypeptides derived from β-cell mRNA as potential source for diabetogenic epitopes. The implication of translational mistakes in autoimmunity demonstrates the need to revise transcriptome based approaches for epitope discovery and offer new foundation for therapeutic approaches focussed on specific induction of immune tolerance. Finally, these findings support the emerging concept that in T1D, β-cells are destroyed by a mechanism comparable to a classical antitumor response where the immune system is perfectly trained and where CTLs and CD4 T cells target dysfunctional cells in which errors have accumulated.

### EXAMPLES

### Example 1. Identification of Peptides Derived from a Defective Ribosomal Product (DRiP) and a Splice Variant in the Human Insulin Gene

To investigate whether epitopes derived from defective ribosomal products (DRiPs) or alternative splicing events could potentially be involved in provoking an autoimmune response to the insulin-producing β-cells, the mRNA of the most abundant β-cell specific preproinsulin (PPI) protein was explored. Using NetStart 1.0 (a prediction algorithm to predict translation initiation sites in eukaryotes; well known to the person skilled in the art) the PPI mRNA was analyzed for alternative translation initiation sites or splicing events. Besides the start codon that is known to give rise to PPI, three other AUG triplets appeared located at position 70, 341 and 442, and one of them (341) was predicted as translation initiation site (FIGS. 1-3). It was envisioned that translation initiation at position 341 could potentially give rise to a polypeptide lacking an in-frame termination codon and is translated in a +2 reading frame compared to PPI, and thus represents a protein with a completely different amino acid sequence. This appeared to be the case. The polypeptide from this start codon (AUG60) can be translated in two isoforms; each having two single nucleotide polymorphism (SNPs; CH and RP, referring to the specific amino acids; see below, bold). Homology comparison of human PPI mRNA with PPI mRNA of other species revealed a human specific G to A point mutation that gave rise to the alternative AUG codon within a strong Kozak consensus sequence, indicating that this indeed concerns an existing to-date unknown human specific non-conventional protein. In addition, a novel splice variant of the human insulin gene was identified, (Splice RP, see below). This aberrant polypeptide is translated as one isoform containing the same SNPs as the INS-DRiP-RP variant.

INS-DRIP-CH:
(SEQ ID NO: 3)
MLYQHLLPLPAGELLQLDAACRQPHTRRLLHRERWNKALEPAK
INS-DRIP-RP:
(SEQ ID NO: 4)
MLYQHLLPLPAGELLQLDAARRQPPTRRLLHRERWNKALEPAK
Splice-RP:
(SEQ ID NO: 5)
MALWMRLLPLLALLALWGPDPAAAFVNQHLCGSHLVEALYLVCGERGFFY
TPKTRREAEDLQAGELLQLDAARRQPPTRRLLHRERWNKALEPAK

The nucleotide sequence (SEQ ID NO:1) and the translated amino acid sequence (SEQ ID NO:2) of the wild type human PPI gene is shown in FIG. 1. One of the four possible versions of the INS-DRiP polypeptide according to the present invention is shown in the +2 reading frame and starts with N-terminal MLYQHLLPL (SEQ ID NO: 8). In FIG. 2, the corresponding mRNA is shown together with both the conventional PPI translation product and one of the four possible versions of the INS-DRiP polypeptide according to the present invention.

In order for the non-conventional polypeptides to be immunogenic (and to play a role in T1D) it needs to generate peptides that efficiently compete with other peptides for presentation on the cell surface by HLA class I and/or class II molecules. To identify potential epitopes encoded by these non-conventional proteins, the amino acid sequences were analyzed with prediction algorithms NetMHC 3.4, SYFPEITHI, BIMAS and MOTIFS, known to the person skilled in the art. Since HLA-A*0201 and -DQ2 and/or -DQ8 are the most prevalent serotype among the T1D population, peptides were screened that would have the potential to bind HLA-A2 or HLA-DQ2 and or DQ8. This led to identification of one potential high-affinity HLA-A2 binding peptide: the first 9 amino acids of the INS-DRiP: MLYQHLLPL (SEQ ID NO: 8). By employing similar in silico studies, also a 9-amino acid potential high-affinity HLA-DQ8cis/trans binding peptide was identified for the Splice RP variant: PTRRLLHRE (SEQ ID NO: 9).

### Example 2. Cell Culture and Transfection

The following methods of cell culture and transfection was applied in the present examples:

HEK 293T Cells:

HEK 293T cells were maintained in Dulbecco's modified Eagle's medium (DMEM) supplemented with 8% heat inactivated FCS, 100 U/ml penicillin and 100 μg/ml streptomycin. HEK 293T cells were transfected in suspension using polyethylenimine (PEI). For transfection of a 6 well, 500 ng pDNA and 1 μg PEI (pH 7.4) was used in a total volume of 50 μl Opti-MEM I reduced serum medium and incubated for 10 min at RT before adding to the cell suspension. After overnight incubation the medium was refreshed and further experiments were performed. For ER stress evaluation, 24 h post transfection, cells were treated with thapsigargin (TG) 2 μM for 6 h.

JY Cells:

JY cells were maintained in IMDM supplemented with 8% FCS, 100 U/ml penicillin and 100 μg/ml streptomycin. For maintenance and expansion autoreactive CD8⁺ T-cell clones were specifically stimulated every 14 days with irradiated allogeneic PBMCs and irradiated peptide-pulsed HLA-A2-expressing JY cells in IMDM supplemented with a mixture of cytokines similarly as described below.

Human Islets:

Human islets were isolated from brain-dead organ donors in the institutional GMP-facility. Purity of the final islet preparation was assessed by 1 mmol/l dithizone (Sigma-Aldrich) staining and ranged from 45-95%. Purified islets were cultured in CMRL-1066 medium supplemented with 10% human serum, 20 μg/ml ciprofloxacin, 50 μg/ml gentamycine, 2 nmol/l L-glutamine, 250 ng/ml fungizone, 10 mmol/l HEPES and 10 mmol/l nicotinamide.

Third generation self-inactivating lentiviral vectors were produced as described previously (Carlotti et al. Lentiviral vectors efficiently transduce quiescent mature 3T3-L1 adipocytes. Mol Ther 9:209-217, 2004). HIV p24 concentration was measured with antigen capture ELISA (ZeptoMetrix, Buffalo, N.Y., USA), 1 ng p24 corresponds to 2500 infectious particles. Human pancreatic islets were dissociated and dispersed prior transduction. The islets were incubated with 0.05% trypsin for 5-10 min at 37° C. and subsequently passed through a 40 μm filter. Immediately after dispersion the islets were transduced with HIP-Luc2CP containing lentivirus at MOI=2. The medium was refreshed after overnight incubation. Cells were maintained in culture for 2 days prior incubation with CTL. Human pancreatic islets were maintained in DMEM low glucose supplemented with 8% heat inactivated FBS, 100 u/ml penicillin and 100 ug/ml streptomycin in ultra-low attachment plates.

MLYQHLLPL-Specific CD8⁺ T-Cell Clones:

The generation and characterization of MLYQHLLPL-specific CD8⁺ T-cell clones was performed as follows: Freshly isolated PBMCs from a long-term HLA-A2⁺ T1D patient were seeded 150,000 cells/well with 10 ug/ml MLYQHLLPL peptide in IMDM supplemented with 10% human serum, 0.5% LeucoA, 0.1 ng/ml IL-12, 10 ng/ml IL-7, 25 U/ml IL-2 and 5 ng/ml IL15. After 14 days of culture, 20,000 cells/well PBMCs were re-stimulated specifically with 20,000 cells/well irradiated MLYQHLLPL peptide-pulsed HLA-A2-expressing JY cells (2 μg/ml peptide with 10×10⁶ cells in AIM-V medium (Life Technologies) for 2 h at 3TC and 100,000 cells/well irradiated allogeneic PBMCs in IMDM medium supplemented with human serum and cytokines as described above. On day 28, CD8⁺ T-cells were double stained with MLYQHLLPL tetramers (tm), single cell sorted into round bottom 96-well plates and re-stimulated as described above. Expanding CD8⁺ tm⁺ T-cell clones were isolated and re-stimulated every 14 days. Every 4-5 days fresh IMDM supplemented with 25 U/ml IL-2 and 10 ng/ml IL-15 was added to the culture media. T-cell characterization was assessed by Flow cytometry using the following markers CD45-FITC, CD3-FITC, CD4-FITC, CD8-FITC, CD16-FITC, CD28-FITC, TCRα/β-FITC, NKG2D-PE (Becton Dickinson, Franklin Lakes, N.J.). Detection of CD8+ T-cell activity: After overnight incubation with this T-cell clone, complete lysis of the target cells (JY) loaded with the MLYQHLLPL peptide was detected in a Chromium release assay at 5:1 effector:target (E:T), while target cells loaded with irrelevant peptide remained unaffected (FIG. 7C). The Chromium release assay were performed as follows: Briefly, JY cells loaded with INS-DRiP₁₋₉ or SP-PPI₁₅₋₂₄ (Signal peptide of the preproinsulin) peptide were incubated with 100 μl Na-chromate (51Cr, 3.7 MBq) and seeded in triplicate for 1 h, at various effector-to-target (E:T) together with CTLs. After 16 h incubation at 37° C. in 5% CO2, supernatants were collected, and the release of 51Cr was measured with a gamma-counter (Wallac/PerkinElmer, Waltham, Mass., USA). Spontaneous and maximum releases were obtained by incubation with medium and 1% triton in PBS, respectively. The specific lysis was calculated as percentage of specific lysis=100×(experimental release−spontaneous release)/(maximum release−spontaneous release). To analyse specific β-cell death, human islet cells were transduced with LV-HIP-Luc2CP lentivirus vector as described previously (31). Transduced islet cells were incubated with increasing amounts with epitope-specific CD8⁺ T-cells in IMDM supplemented with 5% HS, 25 U/ml IL-2 and 10 ng/ml IL-15 for 48 hours. Experiments were performed with different effector islet cells target ratio (E:I ratio) and performed in fourfold. Subsequently, cells were lysed in luciferase lysis buffer (25 mM Tris/HCl pH 7.8, 2 mM CDTA, 2 mM DTT, 10% glycerol, 1% Triton X-100) and T-cell mediated target cell killing was verified by measuring light emission using Luciferase Assay Reagent (Promega) and Lumat LB9501 luminometer (Berthold, Bad Wildbad, Germany). Results are shown as decrease luciferase activity. The percentage of β cell death is calculated using the following formula: 100−[(RLU_{CTLx}/RLU_{average CMV CTL})×100].

Western Blotting:

Protein lysates were analysed on SDS-PAGE. Before loading, samples were boiled in sample buffer (2% SDS, 25 mM Tris-HCl pH 6.8, 1.5 mM Bromophenol blue, 0.14 mM β-mercaptoethanol, 10% glycerol) for 5 min at 96° C. Proteins were transferred to Immobilon-P (Immobilon-P transfer membrane (polyvinylidene difluoride); Millipore) and visualized by standard antibody staining protocols for anti-insulin (1:5000), anti-GFP (1:5000) anti-actin (1:5000) and anti-DRiP/Splice Poab (1:5000).

### Example 3. Binding of Peptide MLYQHLLPL to HLA-A2 and PTRRLLHRE to HLA-DQ—Validation

In a competition-based HLA-peptide binding assay the binding of the newly identified peptide to HLA-A2 was investigated. The MLYQHLLPL (SEQ ID NO: 8) peptide was synthesized in vitro by solid-phase Fmoc chemistry and validated by ultra-performance liquid chromatography and mass spectrometry. Peptides (purity >85%) were dissolved in 5% DMSO/PBS to a 1 mM stock solution and their binding affinity to HLA-A2 was determined by a competition-based cellular peptide binding assay (Kessler et al. Competition-based cellular peptide binding assays for 13 prevalent HLA class I alleles using fluorescein-labeled synthetic peptides. Hum Immunol 64:245-255, 2003). JY cells (HLA-A020) were stripped of their naturally bound peptides in ice-cold citric acid elution buffer (1:1 mixture of 0.263M citric acid and 0.123M Na₂HPO₄, PH 3.1). After 90 seconds incubation, the reaction was stopped by addition of ice-cold Iscove's modified Dulbecco's media (IMDM-2) supplemented with 2% foetal calf serum (FCS). The cell pellet was resuspended in IMDM-2 supplemented with 2% FCS and 2 μg/ml β2M at a concentration of 4×10⁵ cells/ml. An 8-fold dilution series of test peptides (final concentration of 100 μM to 0.8 μM was incubated with fluorescent-labelled reference peptide (150 nM) and 4×10⁴ cells for 24 hrs at 4° C. in the dark. Peptide binding affinity was determined by decrease in fluorescence intensity measured by FACS. The 50% inhibitory concentration (IC50) was calculated by nonlinear regression analysis using the following formula: inhibition (%)=((MF−MFₘᵢₙ)/(MFₘₐₓ−MFₘᵢₙ))*100. The MLYQHLLPL peptide bound with high-affinity (2.3 μM) to HLA-A2 as shown in Table 1.

TABLE 1

| | | Experimental |
|---|---|---|
| Peptide | Sequence | IC₅₀ (nM) |
| HBV cAG₁₈₋₂₇ | FLPSDFFPSV | 1.1 |
| MAGE A wt | YLEYRQVPG | 128.0 |
| INS-DRiP₁₋₉ | MLYQHLLPL | 2.3 |

The results shown in Table 1 shows that the binding affinity of INS-DRiP₁₋₉ to HLA-A2 is in the same range as that of a prototypic viral T-cell epitope. MAGE is a self-protein in melanoma; a target of tumor-specific CTLs, also used as cancer vaccine.

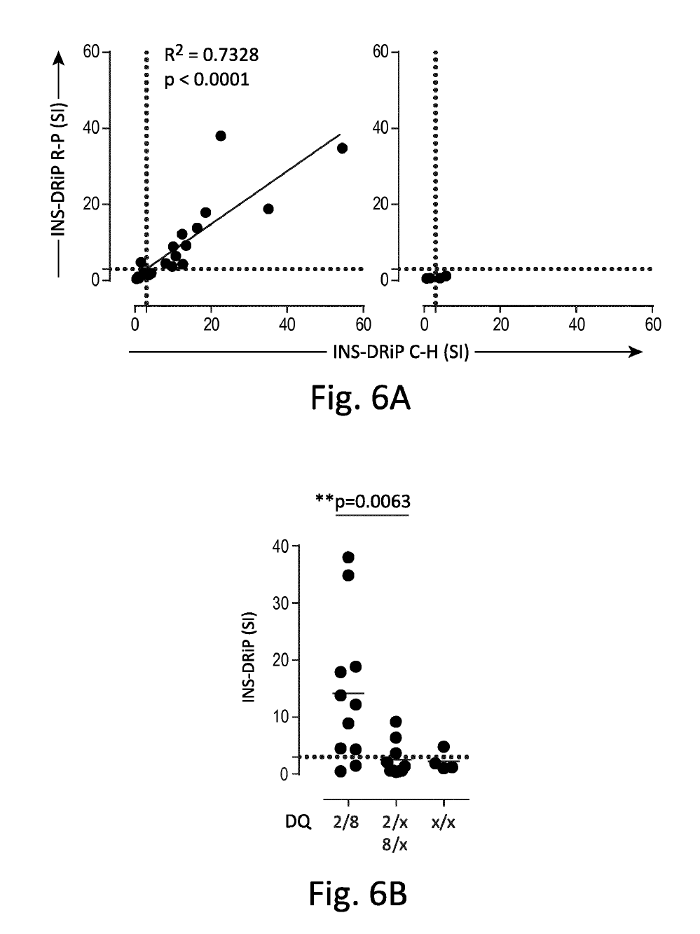

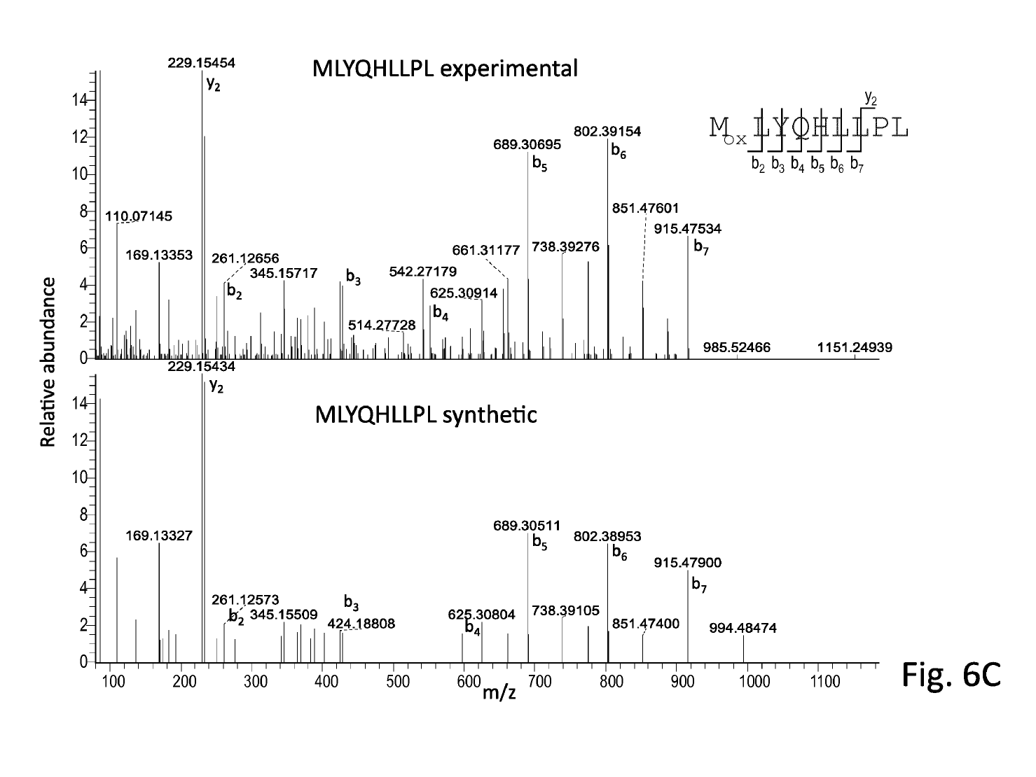

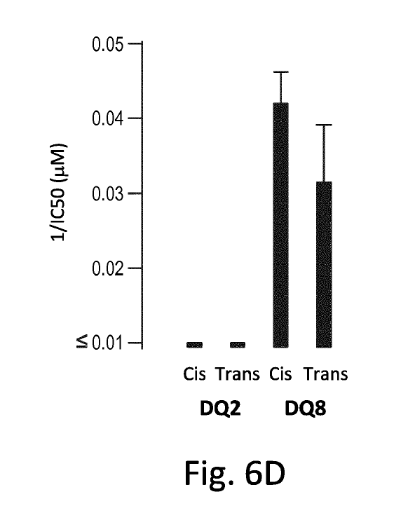

Peptide binding to all four susceptible HLA-DQ molecules was performed in competitive cell-free HLA/peptide binding assays as described previously (van Lummel et al. JBC, 2011; van Lummel et al. Diabetes, 2015). In short, FluoroNunc 96 well plates, coated with SPV-L3 (anti-HLA DQ antibody), were washed and blocked, and were then incubated with whole cell lysates of DQ expressing EBV-BLCL at 4° C. overnight. Titration ranges of test peptides (from 0 to 300 μM) with or without a fixed concentration (0.6 μM) of indicator peptide were applied to the wells containing binding buffer. After incubation, plates were washed and europium-streptavidin in assay buffer was added to each well followed by incubation. After washing, wells were incubated with enhancement buffer. Plates were read using a time-resolved fluorometer (Victor, Wallac). EC₅₀ values were calculated based upon the observed binding of the test peptides against the fixed concentration indicator peptide; the concentration of test peptide required for half-maximal inhibition of binding of the reporter peptide indicate the EC₅₀ value. Binding of the naturally processed peptide MLYQHLLPL eluted from DQ2/8 DCs was confirmed for DQ8cis and DQ8trans (see example 6 and FIG. 6D); the reciprocal EC₅₀ value is provided here showing that larger bars represent higher affinity of the MLYQHLLPL peptide for DQ. Binding of this peptide was not observed for the DQ2cis/trans molecules. Binding of the in silico predicted PTRRLLHRE (SEQ ID NO: 9) peptide was confirmed for HLA-DQ8cis/trans (data not shown). No binding of the peptides to the HLA-DQ2cis/trans molecules was observed.

### Example 4. Preparation of GFP Constructs and Expression of GFP Fusion Proteins

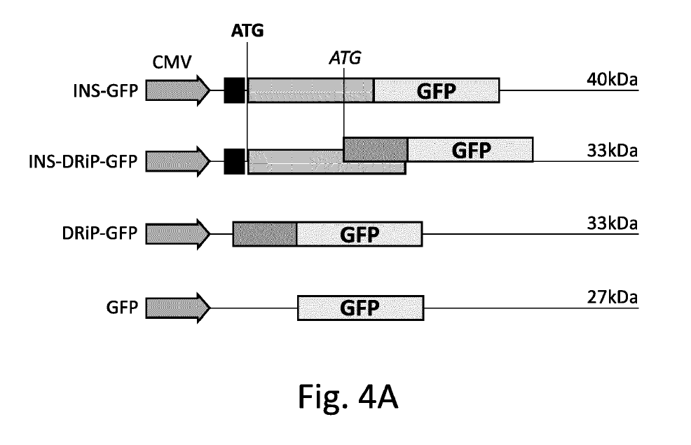

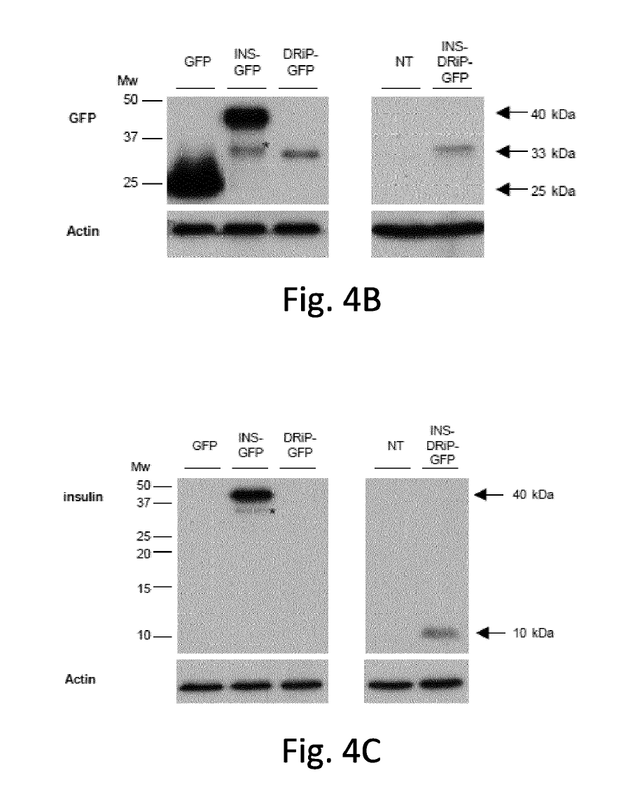

In order to validate the functionality of the different AUGs, GFP fusion constructs were constructed where the GFP coding sequence was cloned in frame with AUG60 or AUG341 generating two fusion proteins, INS-GFP (also called PPI-GFP) and INS-DRiP-GFP (also called PPI/DRiP-GFP), respectively (FIG. 4A). The cloned insulin gene encoded the INS-DRiP-RP isoform. As control, the DRiP-GFP fragment was cloned out of the insulin mRNA context immediately after the CMV promoter (CMV-DRiP-GFP). The canonical PPI AUG is shown in bold, AUG341 is shown in italic. The expected size of the GFP protein and the fusion variants are depicted on the right.

GFP fusion constructs were generated using methods and general knowledge known to the person skilled in the art. pCMV-PPI-GFP and pCMV-PPI-DRiP-GFP were generated from the intermediate cloning vectors pJET1.2-PPI and pJET1.2-DRiP, respectively. pJET1.2-PPI was generated by cloning PPI (5′-UTR to PPI termination codon) into pJET2.1/blunt vector using the primers: Fw 5′-AGCCCTCCAGGACAGGC-3′ (SEQ ID NO: 16) and Rv 5′GTTGCAGTAGTTCTCCAGCT-3′ (SEQ ID NO: 17) on human cDNA. pJET1.2-DRiP was generated by cloning PPI (5′-UTR to 3′-UTR) into pJET2.1/blunt vector using the primers: Fw 5′-AGCCCTCCAGGACAGGC-3′ (SEQ ID NO: 16) and Rv 5′-TTTTGCTGGTTCAAGGGCTTTATT-3′ (SEQ ID NO: 18) on human cDNA. The Insulin and DRiP insulin fragment were sub-cloned into pEGFP-N1 (Clontech) in-frame with the GFP ORF to generate PPI-GFP and DRiP-GFP expressing vectors.

Following transfection in HEK 293T cells, both INS-GFP (also called PPI-GFP) and INS-DRiP-GFP (also called PPI/DRiP-GFP) constructs led to expression of GFP fusion proteins migrating at 40 kDa and 33 kDa, respectively (FIGS. 4B and 4C, left panel). The control construct CMV-DRiP-GFP confirmed the molecular weight of the unconventional product (FIG. 4C, right panel). As expected, cells transfected with INS-DRiP-GFP also co-express proinsulin as assessed by western blot (FIG. 4D) and immunohistochemistry (data not shown).

Transfection of HEK 293T cells and western blotting of the cell lysates was performed as described in example 2.

### Example 5. Effect of ER Stress on Translation Initiation

In order to determine the effect of endoplasmic reticulum stress (ER stress) on translation initiation, INS-DRiP-GFP transfected HEK 293T cells were stimulated with the ER stress inducers thapsigargin (TG) or tunicamycin (TM). For ER stress evaluation, 24 h post transfection, cells were treated with thapsigargin 2 μM for 6 hours or tunicamycin for 5 h 2 uM was used for 6 h.

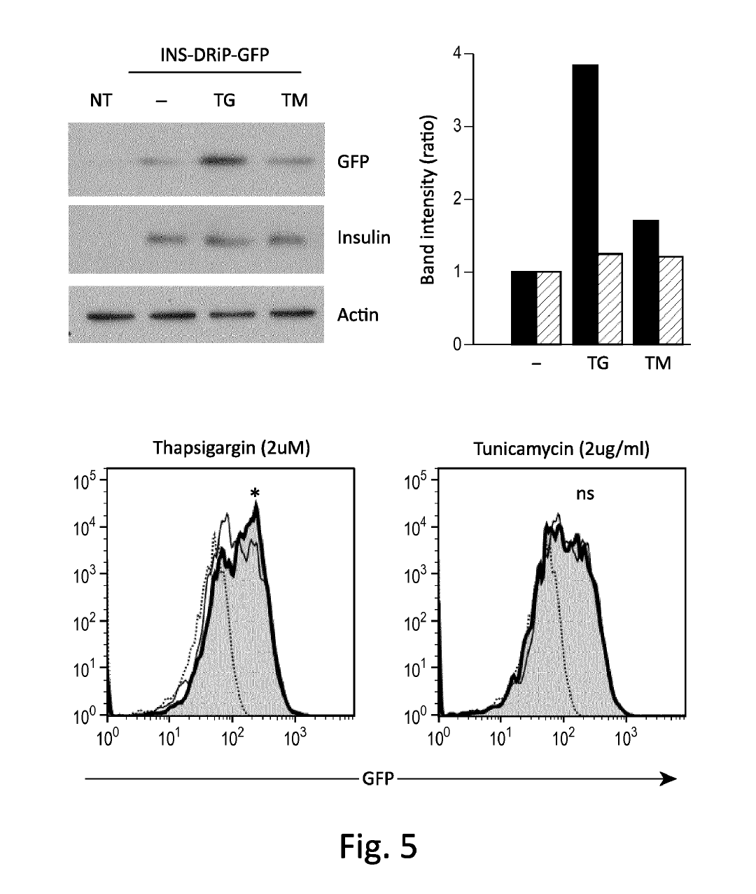

It follows from the results obtained that the expression of INS-DRiP polypeptide was increased by TG treatment as quantified both by western blot (FIG. 5, upper panel) and flow cytometry analysis (FIG. 5, lower panel). Altogether these data indicate that ER stress contributes to expression of the INS-DRiP polypeptide similarly as to other stress-induced proteins.

### Example 6. T Cell Proliferative Response to the Aberrant INS-DRiP-CH and INS-DRiP-RP Polypeptides

Freshly isolated PBMCs were used to investigate the immunogenicity of recombinant INS-DRiP polypeptide in a T-cell proliferation assay. PBMCs were seeded (150,000/well) in flat-bottomed 96-well microculture plates (Greiner, Nürtingen, Germany) and cultured for 5 days at 37° C. in 5% CO2, in a humidified atmosphere. Cells were cultured in triplicates in medium alone, with 10 μg/mL recombinant INS-DRiP polypeptide, or recombinant IL-2 10% (25 units/mL; Genzyme, Cambridge, Mass.) as positive control. In the final 16 h of culture, 50 μL RPMI 1640 (Dutch modification; Gibco) containing 0.5 μCi 3H-thymidine (DuPont NEN, Boston, Mass.) was added per well. After the cells were harvested on filters with an automated harvester, proliferation was determined by the measurement of 3H-thymidine incorporation in an automatic liquid scintillation counter. All results are calculated as mean counts per minute (CPM) in the presence of antigen and compared with medium alone.

Recombinant INS-DRiP-RP and INS-DRiP-CH polypeptides were obtained from human islet cDNA by PCR, consisting of the 43 amino acids encoded within the insulin mRNA excluding the poly-A tail. subsequently cloned into a bacterial expression vector Gateway cloning technology (Invitrogen, Carlsbad, Calif., USA) creating an N-terminal histidine tag, used for protein purification on nickel column (GE Healthcare, #17531801). Peptides were synthesized by solid-phase Fmoc chemistry and validated by ultra-performance liquid chromatography and mass spectrometry. Peptides (purity >85%) were dissolved in 5% DMSO/PBS to a 1 mM stock solution.

FIG. 6 shows T cell responses within PBMCs isolated from T1D patients against the aberrant INS-DRiP-RP or INS-DRiP-CH polypeptides. Proliferative responses in presence of the INS-DRiP-RP and INS-DRiP-CH recombinant polypeptides is shown as the stimulation index (SI) calculated by dividing the mean CPM in presence of protein by the mean CPM of medium alone; SI≥3 are considered as positive. In FIG. 6A, high T-cell proliferative responses against the INS-DRiP-RP or INS-DRiP-CH polypeptides were observed in T1D patients carrying the high-risk INS-DRiP-RP variant (left), whereas patients carrying the protective DR15/DQ6 haplotype or the protective DRiP CH variant responded only to the INS-DRiP-CH polypeptide (right). Patients heterozygous for DQ2 and DQ8 (highest susceptible risk for T1D) show higher proliferative responses to the INS-DRiP variants as compared to patients carrying either DQ2 or DQ8. FIG. 6B shows proliferative responses against the two INS-DRiP proteins of PBMC isolated from T1D patients carrying the highest risk HLA-DQ2/8 genotype (DQ2/DQ8) compared to patients carrying DQ2/x or DQ8/x and compared to patients carrying neither HLA-DQ2 nor HLA-DQ8 (x/x). Patients heterozygous for DQ2 and DQ8 show higher proliferative responses to the INS-DRiP variants as compared to patients carrying either DQ2 or DQ8. FIG. 6C shows HLA-DQ peptidome analysis of dendritic cells (DCs). Immature DCs, heterozygous for HLA-DQ2/8 were pulsed with the INS-DRiP polypeptides. After maturation, HLA-DQ molecules were purified from the lysed pulsed DCs and the naturally processed and HLA-bound peptides were acid eluted. Using state-of-the-art mass spectrometry (van Lummel, Diabetes, 2015) a peptide with the sequence MLYQHLLPL (SEQ ID NO: 8) derived from the N-terminus of the INS-DRiP variants was identified. The experimental spectrum (top) of this MLYQHLLPL peptide, now identified as a naturally processed peptide eluted from highest-risk HLA-DQ2/8 heterozygous DCs, fully matches the spectrum of the same synthesized peptide (bottom). In FIG. 6D binding of the MLYQHLLPL peptide was confirmed in cell-free HLA-DQ peptide binding assays (van Lummel, J B C, 2011). Binding of MLYQHLLPL was observed for the T1D highest-risk DQ8cis and DQ8trans molecules. Binding to T1D low-risk DQ2cis/trans was not observed.

### Example 7. Detection of Epitope-Specific CD8+ T-Cells in T1D

In order to assess the clinical relevance of the INS-DRiP₁₋₉ (SEQ ID NO: 8) epitope (also called the MLYQHLLPL epitope) by exploring the presence of specific CD8 T-cells in PBMC samples obtained from T1D HLA-A2⁺ patients and HLA and age matched healthy donors. The detection of MLYQHLLPL-specific CD8⁺ T-cells in PBMCs of T1D patients was performed as follows. Heparinized blood samples were drawn from 14 T1D patient (age range 8-23 years; disease duration 5-30 months) and 14 sex- and age-matched healthy controls. Peripheral blood mononuclear cells (PBMCs) were isolated by Ficoll-isopaque density gradient centrifugation and frozen in liquid nitrogen until use. For the detection of MLYQHLLPL-specific CD8⁺ T-cells, thawed PBMCs were stained with Qdot-labeled multimeric complexes. Peptide-HLA-A2 (pHLA-A2) monomers and multimeric pHLA-A2 complexes were generated as previously described (Abreu et al. CD8 T cell autoreactivity to preproinsulin epitopes with very low human leucocyte antigen class I binding affinity. Clin Exp Immunol 170:57-65, 2012). Samples with CD8+ T-cell counts under 50,000 were excluded from analysis.

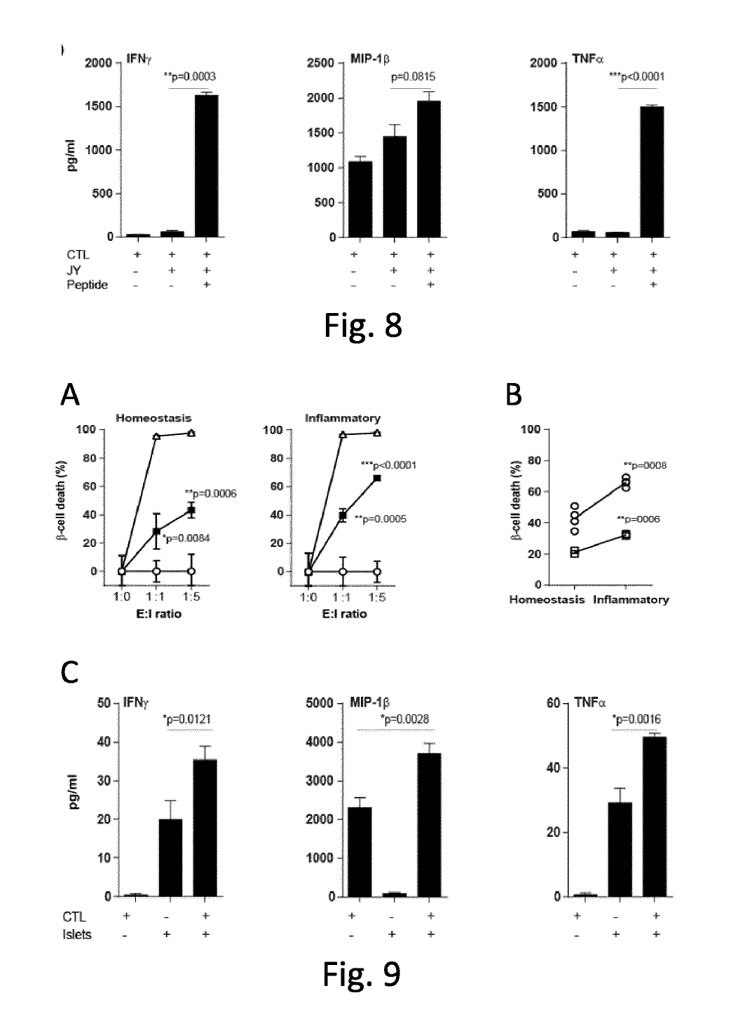

The results are provided in FIG. 7. As shown in FIG. 7A, significantly higher levels of INS-DRiP₁₋₉-specific CD8+ T cells (left) in T1D patients were detected whereas antiviral CD8+ T-cell frequencies were comparable between the T1D and healthy donors (right). P values were calculated by unpaired Student's t-test; NS=not significant. To further characterize these T-cells, INS-DRiP₁₋₉-specific T-cell clones were generated from PBMCs isolated from T1D patients by dual tetramer staining and their cytotoxic properties were determined on peptide loaded HLA-A2+ JY cells (FIG. 7B, 7C, 7D). Although the isolated clones have different TCR sequences, complete lysis of peptide-pulsed target JY cells was detected, whereas target cells loaded with irrelevant peptide remained unaffected (FIG. 7D). FIG. 7B shows a T-cell clone which was generated from PBMCs isolated from a long-term HLA-A2+ T1D patient. After 28 days in culture with periodic peptide specific re-stimulation, CD8+ Tm+ cells were detected (upper panel), flow-sorted and seeded 1 cell/well. MLYQHLLPL-directed CTL clones were validated by dual tetramer (using APC and PE as fluorescent labels) staining (lower panel). After overnight incubation with this T-cell clone, complete lysis of the target cells (JY) loaded with the MLYQHLLPL peptide was detected in a Chromium release assay at 5:1 effector:target (E:T), while target cells loaded with irrelevant peptide remained unaffected (FIG. 7C). The chromium release assay was performed as described in the above. Furthermore, and as shown in FIG. 8, MLYQHLLPL-specific T-cell activation resulted in a significant increase in secretion of the proinflammatory cytokines IFNγ and TNFα and to a lesser extent MIP-1β, when compared to non-stimulated T-cells. P values were calculated by unpaired Student's t-test.

### Example 8. Diabetogenic Potential of INS-DRiP₁₋₉-Specific CTLs

The diabetogenic potential of the INS-DRiP₁₋₉-specific CTL was investigated by assessing the cytotoxic potential of MLYQHLLPL-specific CTL on HLA-A2 expressing human islet cells. In order to specifically investigate β-cells, dispersed primary human islets were transduced by lentivirus containing the β-cell specific viability reporter LV-HIP-LUC2CP as previously described (Zaldumbide, Mol Ther, 2013). The LV-HIP-LUC2CP viability reporter is a short half-life luciferase reporter gene under the control of the human insulin promoter. Transduced human pancreatic islet cells were incubated with CTLs specific for MLYQHLLPL (INS-DRiP₁₋₉), CMVpp65 or PPI₁₅₋₂₄ in IMDM supplemented with 5% HS, 25 U/ml IL-2 and 10 ng/ml IL-15 for 48 hours. Experiments were performed with different effector islet cells target ratio (E:I ratio) and performed in fourfold. Subsequently, cells were lysed in luciferase lysis buffer (25 mM Tris/HCl pH 7.8, 2 mM CDTA, 2 mM DTT, 10% glycerol, 1% Triton X-100) and T-cell mediated target cell killing was verified by measuring light emission using Luciferase Assay Reagent (Promega) and Lumat LB9501 luminometer (Berthold, Bad Wildbad, Germany). Results are shown as decrease luciferase activity. The percentage of β cell death is calculated using the following formula: 100−[(RLU_{CTLx}/RLU_{average CMV CTL})×100].

In order to test whether inflammation increased beta cell susceptibility to cytolysis by peptide-specific T-cells, the Luciferase killing assay was performed on the dispersed and HIP-Luc2CP transduced human islet cells maintained in normal low glucose media (5 mM) (FIG. 9A, left) or high glucose combined with proinflammatory cytokines IL-1β and IFN-γ to mimic T1D pathology (20 mM glucose, IFN-γ and IL1β) (FIG. 9A, right). As can be seen from FIG. 9B, these stress conditions further increased β-cell death in the presence of MLYQHLLPL specific CTLs as shown by the percentage of β-cell death observed in two different pancreas preparation after incubation with MLYQHLLPL-specific CTL in homeostatic or inflamed condition (E:I ratio 1:5). Results are shown as % of β-cell death (% of decreased luciferase) after 48 h after co-culture with PPI-directed CTL (open triangles), pp65CMV directed CTL (open circles) or MLYQHLLPL-directed CTL (black squares). P values were calculated by unpaired Student's t-test relative to pp65CMV directed CTL. The results shown in FIG. 9B) indicates that DRiP epitope presentation is enhanced in inflammatory conditions.

As expected, PPI₁₅₋₂₄-specific CTLs destroyed β-cells as reflected by the decrease in luciferase activity, whereas CMVpp65-directed CTLs had no effect on β-cells. It was found that INS-DRiP₁₋₉-specific CTLs significantly reduced β-cell survival. The β-cell destruction by the INS-DRiP₁₋₉-specific CTLs confirmed that the DRiP₁₋₉ (MLYQHLLPL) epitope is naturally generated, processed and presented on the cellular surface on human β-cells. The partial destruction of β cell mass by INS-DRiP₁₋₉-specific CTL (CTL targeting MLYQHLLPL) is in tune with the error hypothesis and points to β-cell heterogeneity and immunogenicity perhaps due to differential sensitivity to stress factors.

In order to assess the specific stimulation and activation of the CD8+ T-cells by islets cells, cytokine production by INS-DRiP₁₋₉-specific CTLs after co-culture with HLA-A2+ human islet cells. IFN-γ, TNF-α and MIP-1β were measured after 48 h incubation in low glucose culture conditions. P value was calculated by unpaired Student's t-test relative to cytokines released by islets in absence of T-cells (IFN-γ and TNF-α) or cytokines released by CTL in absence of islets (MIP-1β). As can be seen in FIG. 9C, when co-cultured with human islets, INS-DRiP₁₋₉-specific CTLs secreted effector cytokines MIP-1β, IFNγ and TNFα, indicating the specific stimulation and activation of the CD8+ T-cells by islets cells.

Without wishing to be bound to any theory, our results support the emerging concept that beta cells are destroyed in T1D by a mechanism comparable to classical antitumor responses where the immune system has been trained to survey dysfunctional cells in which errors have accumulated and offer alternatives for tissue and antigen specific therapeutic approaches aiming at the induction of immune tolerance.

### Example 9. Peptide-Induced Production of Granzyme B by CD8+ T-Cells

Detection of Granzyme B (GrzB) production by CD8 T cells in response to the MLYQHLLPL peptide was performed using an enzyme-linked immunospot (ELISPOT), as described previously (Arif et al. Autoreactive T cell responses show proinflammatory polarization in diabetes but a regulatory phenotype in health. J Clin Invest 113:451-463, 2004; Herold et al. Validity and reproducibility of measurement of islet autoreactivity by T-cell assays in subjects with early type 1 diabetes. Diabetes 58:2588-2595, 2009). After informed consent, blood was collected from 35 T1D patients. PBMC from celiac disease (CD) patients were used as control subjects (n=5). PBMCs were freshly isolated and incubated in the presence of 10 μg/mL peptide or diluent alone for 48 h. The cells were then transferred onto micro-titer plates pre-coated with monoclonal anti-GrzB capture antibody. Detection of cells producing GrzB was performed using biotinylated anti-GrzB detector antibody. Fourteen patients (14/35; 40%) responded to the epitope tested by production of granzyme B (GrzB) and/or IFN-γ (data not shown). Healthy subjects did not respond to the MLYQHLLPL epitope. Detailed analysis of the cytokine production of INS-DRiP₁₋₉-specific CD8 T-cells revealed a response dominated by GrzB production in T1D patients as compared to control subjects (data not shown).

### Example 10. Preparation of Polyclonal Antibody Against Peptide Present in DRiP/Splice Variants

The anti-DRiP/Splice Poab was prepared by raising polyclonal rabbit antiserum against amino acids 30-43 of the DRiP variants (homologous to amino acid 82-95 of the Splice variant), namely LHRERWNKALEPAK (SEQ ID NO:7), or immunogenic fragments therein.

### Example 11. Immunohistochemistry with a Polyclonal Serum Raised Against a Peptide within the INS-DRiP Variants and the Splice-RP Variant

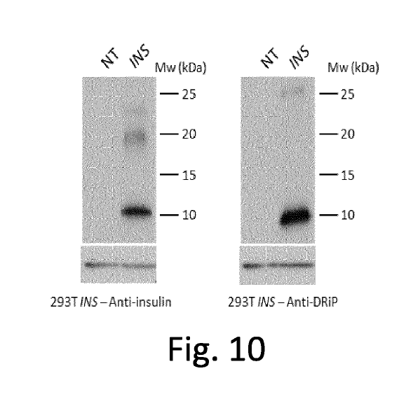

HEK 293T cells were transfected with the constructs encoding the normal insulin products or the aberrant insulin DRiP in suspension using polyethylenimine (PEI). For transfection of a 6 well, 500 ng pDNA and 1 μg PEI (pH 7.4) was used in a total volume of 50 μl Opti-MEM I reduced serum medium and incubated for 10 minutes at RT before adding to the cell suspension. After overnight incubation the medium was refreshed and further experiments were performed. Protein lysates of transfected HEK cells were analysed on SDS-PAGE. Before loading samples were boiled in sample buffer (2% SDS, 25 mM Tris-HCl pH 6.8, 1.5 mM Bromophenol blue, 0.14 mM β-mercaptoethanol, 10% glycerol) for 5 minutes at 96° C. Proteins were transferred to Immobilon-P (Immobilon-P transfer membrane (polyvinylidene difluoride); Millipore, Etten-Leur, The Netherlands) and visualized by standard antibody staining protocols for anti-insulin (1:5000) showing a product of 11 kDa (FIG. 10, left) or anti-DRiP/Splice Poab (1:5000) showing a product of 9 kDa (FIG. 10, right). Although the Poab does not discriminate between the INS-DRiP variants and the Splice-RP variant, these results show that the human insulin gene encodes an alternative polypeptide.

It is apparent from FIG. 10 that transfection of HEK cells with the normal INS gene or the aberrant INS gene result in translation of normal insulin (FIG. 10, left) and an aberrant insulin product (FIG. 10, right) as visualized by western blot and staining with anti-insulin or anti-DRiP/Splice. Immunohistochemical stainings on three paraffin embedded pancreatic sections staining with anti-insulin antibody, DAPI (nuclear staining) and anti-DRiP/Splice Poab, showed that the both the INS gene and an aberrant INS gene is present in all of these three sections. These results demonstrate that human pancreatic islets express the Splice variant and/or a INS-DRiP variant. No staining was observed in exocrine tissue. The results show staining of three different islets (from the same donor) illustrating heterogeneity of expression of this polypeptide (low, intermediate and high expression phenotype). As the Poab is generated against a sequence present in both the INS-DRiP variants as the Splice variant, it is yet inconclusive which aberrant insulin product(s) is expressed.

### Example 12. Immunotherapy

The investigational medicinal product consists of dendritic cells generated from autologous monocytes that have been modulated in culture to display a tolerogenic phenotype and function (TolDC), and pulsed with synthetic INS-DRiP₁₋₉ (INS-DRiP₁₋₉TolDC).

Generation of INS-DRiP₁₋₉

The synthetic INS-DRiP1-9 is manufactured at the Peptide Laboratory of the Interdivisional GMP-Facility of Leiden University Medical Center (LUMC; IGFL). Please refer to the Investigation on Medicinal Product Dossier (IMPD) on INS-DRiP₁₋₉ for detailed information on production, laboratory details and release criteria.

Generation of Autologous INS-DRiP1-9-Pulsed TolDC (PlpepTolDC)

The manufacturing process is performed at the Center for Stem Cell therapy (CST) and at the Interdivisional GMP facility of the LUMC (IGFL). At the CST, which is JACIE/CCKL accredited (NL-022-2010), the isolation of CD14+ cells from the leukapheresis product is performed. At the IGFL, which holds an EU manufacturing license for the aseptic production of Investigational Advanced Therapy Medicinal Products (NL/H 12/0020), the remaining (major part) of the production is performed. The production at the IGFL is performed in a class B cleanroom environment of the IGLF in a class A Laminair Airflow (LAF) cabinet (class A).

Generation of autologous INS-DRiP₁₋₉TolDCs is briefly summarized as follows:

Day 0: CD14+ cells are isolated from the leukapheresis product using the CD14 CliniMACS reagent. The CliniMACS CD14 Product Reagent consists of murine anti-CD14 monoclonal antibodies conjugated to superparamagnetic iron dextran particles. Labeled CD14+ cells are isolated on a CliniMACS Instrument using specifically designed software. The positive fraction after CD14 isolation are washed and suspended in culture medium (RPMI containing 10% FBS, 500 IU/ml penicillin and 500 μg/ml streptomycin and 2 mM Glutamin). CD14+ cells are cultured at a concentration of 0.8×106/ml in culture medium adjusted with 500 U/ml IL-4, 800 U/ml GM-SCF and 1×10-8M Vitamin D3 (Calcitriol).

Day 3: Culture medium is refreshed. Half of the medium is removed from the culture bag and the same volume of the fresh culture medium with 1000 U/ml IL-4, 1600 U/ml GM-SCF, 2×10-8 M Vitamin D3 and 2×10-6M Dexamethason is added to the culture.

Day 6: Immature (iDC) are harvested and cryopreserved in 10% DMSO in a temperature-controlled cryo-vessel and cells are stored in the gas phase of liquid N2 tank.

Day −2 before administration: The immature TolDC are thawed and matured by culturing in culture medium containing 800 IU/ml GM-CSF, 335 IU/ml TNFα, 500 IU/ml IL-6, 1600 IU/ml IL-1β and 2 μg/ml PGE2 at a cell concentration of 0.5×106/ml.

Day of the administration to the patient: Mature TolDCs are loaded with INS-DRiP₁₋₉. Peptide loading is performed at a cell concentration of 0.5×106/ml, with 10 ug/ml (app. 4 uM) peptide for 4 hrs at 37° C. In the meantime, the quality control of mature TolDCs for release takes place. Mature TolDCs that meet release criteria are harvested and prepared for administration. Administration of products that deviate from the release criteria is allowed after a joint risk analysis between the patients' physicians, the pharmacist and the principal investigator and after full renewed consent of the patient to proceed with the study.

Preparation for Administration to the Patient

INS-DRiP₁₋₉TolDCs are washed and suspended in a solution of NaCl 0.9% supplemented with 0.5% human albumin (vehicle). The product is packaged in luer lock syringes in a concentration of 5×106 cells/ml, manually closed with a syringe cap, and labelled. The final individual preparation is performed in a Laminar Airflow Cabinet (class A) in a class B cleanroom of the IGFL. After preparation the product is transported to the patient ward for administration. For detailed information on production, laboratory details and release criteria, reference is made to the Investigation on Medicinal Product Dossier on INS-DRiP₁₋₉TolDCs.

Preparation and Labeling of Investigational Medicinal Product

The preparation of the INS-DRiP₁₋₉TolDC product is performed as described above and according to GMP guidelines. Labelling of the INS-DRiP₁₋₉TolDC-product is performed according to the annex 13 EU GMP guideline and the ISBT128 standard for human blood and tissue products. In practice per administration a sealed bag thus labelled contains up to four 1 ml containing syringes with the INS-DRiP₁₋₉TolDC single cell suspensions, which in their turn because of their small size is labelled with a smaller label.

Leukapheresis Procedure

In order to obtain monocytes for culture of autologous INS-DRiP₁₋₉TolDCs, the patient must undergo a leukapheresis procedure using two peripheral venous access lines. Each patient undergoes a single leukapheresis that obtains sufficient number of monocytes to generate immature TolDCs, which are stored in the liquid nitrogen until further use for the active treatment. The minimum number of mononuclear cells (MNC) to be harvested is 7×109, to allow for two injections of the highest (20×106) INS-DRiP₁₋₉TolDC dose. For the majority of donors (90%), this number of MNC is known to be obtainable in a 2.5 hours leukapheresis procedure. The leukapheresis procedure is performed at least 15 days and not longer than 39 weeks prior to the first planned administration of INS-DRiP₁₋₉TolDCs. Prior to the leukapheresis procedure, the patient is subjected to a standard limited screening procedure consisting of medical history, physical examination, hematology, chemistry, and viral screening, as described in paragraph 10. The minimal 15-day period between leukapheresis and first administration of INS-DRiP₁₋₉TolDCs is required to ensure i) sufficient time for the patient to recover from leukapheresis and ii) to allow for completion of the INS-DRiP₁₋₉TolDC culture. A maximum of 39 weeks between leukapheresis and first administration of INS-DRiP₁₋₉TolDCs, is the period for which the quality of cryopreserved immature TolDCs is guaranteed.

Treatment

Study patients are allocated to a treatment group Dose 1 (D1), Dose 2 (D2), or Dose 3 (D3). Each enrolled patient first undergoes a single leukopheresis procedure and subsequently receives 2 sets of intradermal injections with a 28 day interval consisting of autologous cryopreserved INS-DRiP₁₋₉TolDCs in vehicle (D1=0.5×107, D2=1×107 or D3=2×107 INS-DRiP₁₋₉TolDCs per set of injections). Dose escalation requires safety evaluation after at least 12 weeks of follow up of all patients in the preceding cohort. The first patient of each dose cohort functions as metabolic control of the study protocol by receiving first one set of intradermal injections with vehicle only; these patients receiving vehicle injections is followed for 12 weeks and thereafter continue as soon as possible with the INS-DRiP₁₋₉TolDCs injections and the 24 week follow up. The day of the first INS-DRiP₁₋₉TolDC administration is defined as day 0.

### Embodiments of the Invention

The present invention also relates to the following embodiments:
1. An isolated peptide representing a T-cell epitope present in a Defective Ribosomal Product (DRiP) from the human preproinsulin (PPI) gene.
2. A peptide according to embodiment 1, wherein said DRiP is translated from an out-of-frame AUG start codon within said human PPI gene.
3. A peptide according to embodiment 1 or 2, wherein said DRiP comprises the amino acid sequence of SEQ ID NO:3 or SEQ ID NO:4.
4. A peptide according to embodiment 3, comprising the amino acid sequence MLYQHLLPL of (SEQ ID NO: 8).
5. An isolated peptide representing a T-cell epitope present in a splice variant polypeptide from the human PPI gene.
6. A peptide according to embodiment 5, wherein said splice variant polypeptide comprises the amino acid sequence of SEQ ID NO:5.
7. A peptide according to embodiment 6, comprising the amino acid sequence PTRRLLHRE of (SEQ ID NO: 9).
8. A peptide according to any one of embodiments 1 to 7, wherein said peptide is produced in vitro.
9. A composition comprising a peptide according to any one of embodiments 1 to 8, and a culture of tolerance promoting cells.
10. A composition comprising a peptide according to any one of embodiments 1 to 8, and a tolerance promoting adjuvant.
11 An isolated peptide according to any one of embodiments 1 to 8, or a composition according to claim 9 or 10, for use in the prevention, diagnosis or treatment of type 1 diabetes mellitus.
12. Use of a peptide according to any one of embodiments 1 to 8 in the manufacture of a medicament for the prevention, diagnosis or treatment of type 1 diabetes mellitus.
13. Method of treating a human subject having type 1 diabetes mellitus, or that is at risk of developing type 1 diabetes mellitus, said method comprising administering to said human subject a peptide according to any one of embodiments 1 to 8, or a composition according to embodiment 9 or 10.
14. An antibody or functional fragment thereof, or a polyclonal antiserum, raised and/or directed against:
   a DRiP of SEQ ID NO:3 or 4; or
   a splice variant of PPI, wherein said splice variant comprises the amino acid sequence of SEQ ID NO:5.
15. A polyclonal antiserum according to embodiment 14, wherein said polyclonal antiserum is raised and/or directed against the peptide of SEQ ID NO:7.
16. Use of a polyclonal antiserum according to claim 15 in the diagnosis of type 1 diabetes mellitus.
17. An isolated nucleic acid encoding the peptide of any one of embodiments 1 to 8.

1. A polypeptide or peptide comprising an epitope present in a Defective Ribosomal Product (DRiP) from the human PPI mRNA.

2. A polypeptide or peptide according to claim 1, wherein
a) said DRiP is translated from an out-of-frame ATG start codon within said human PPI mRNA;
b) said DRiP comprises the amino acid sequence of SEQ ID NO: 3 or SEQ ID NO: 4;
c) the epitope comprises at least 5 consecutive amino acids, such as at least 6, at least 7, at least 8, or at least 9 consecutive amino acids, present in said DRiP;
d) the epitope comprises an amino acid sequence selected from the group consisting of LHRERWNKALEPAK (SEQ ID NO: 7) and MLYQHLLPL (SEQ ID NO: 8);
e) the epitope is a T-cell epitope; and/or
f) said polypeptide or peptide is isolated.

3-8. (canceled)

9. A polypeptide or peptide as defined in claim 1, and/or a cytotoxic T-cell targeting said polypeptide or peptide, for use
a) in the diagnosis of type 1 diabetes mellitus; or
b) as a biomarker for type 1 diabetes mellitus and/or in detecting β-cell stress.

10. (canceled)

11. An isolated nucleic acid encoding a polypeptide or peptide as defined in claim 1 or the complementary nucleic acid thereof, or an expression vector comprising the nucleic acid.

12. (canceled)

13. An isolated peptide-MHC complex, wherein the peptide forming part of the complex is a polypeptide or peptide as defined in claim 1, or an isolated binding molecule capable of specifically binding i) the peptide-MHC complex or ii) the polypeptide or peptide.

14. (canceled)

15. An isolated binding molecule according to claim 13, wherein
a) the isolated binding molecule is an isolated antibody; and/or
b) the isolated binding molecule comprises complementarity determining regions derived from a TCR capable of specifically binding the peptide-MHC complex.

16. (canceled)

17. A polyclonal antiserum raised and/or directed against a polypeptide or peptide as defined in claim 1.

18. An isolated binding molecule as defined in claim 13, or a polyclonal antiserum raised and/or directed against the polypeptide or peptide, for use in the diagnosis of type 1 diabetes mellitus, in in vivo and in vitro imaging, for drug targeting, or in detecting β-cell stress.

19. (canceled)

20. A method for identifying a subject suffering from, or being at risk of suffering from, type 1 diabetes mellitus, the method comprising:
a) measuring the amount of polypeptide or peptide as defined in claim 1 in a blood, serum or plasma sample from said subject; and
b) comparing the measured amount of said polypeptide or peptide to a reference value,
wherein a significant deviation in the amount of measured polypeptide or peptide compared to the reference value is indicative of type 1 diabetes mellitus.

21. A method according to claim 20, the method comprising using an isolated antibody capable of specifically binding the polypeptide or peptide, or a polyclonal antiserum raised and/or directed against the polypeptide or peptide, for measuring the amount of said polypeptide or peptide.

22. A method for identifying a subject suffering from, or being at risk of suffering from, type 1 diabetes mellitus, the method comprising:
a) measuring the amount of cytotoxic T-cells targeting a polypeptide or peptide as defined in claim 1 in a blood sample from said subject; and
b) comparing the measured amount of said cytotoxic T-cells to a reference value,
wherein a significant deviation in the amount of measured cytotoxic T-cells compared to the reference value, is indicative of type 1 diabetes mellitus.

23. A method according to claim 22, the method comprising using an isolated peptide-MHC complex, wherein the peptide forming part of the complex is the polypeptide or peptide, for measuring the amount of said cytotoxic T-cells.

24. A culture of tolerance promoting cells targeting a polypeptide or peptide as defined in claim 1.

25. A culture of tolerance promoting cells according to claim 24, wherein said tolerance promoting cells are selected from the group consisting of immature dendritic cells and dendritic cells.

26. A composition comprising an isolated polypeptide or peptide as defined in claim 2.

27. A composition according to claim 26, wherein
a) the composition further comprises a culture of tolerance promoting cells targeting the polypeptide or peptide;
b) the composition further comprises a tolerance promoting adjuvant; and/or
c) the composition is a pharmaceutical composition.

28-29. (canceled)

30. A composition as defined in claim 26, for use
a) as a medicament;
b) in a method for the prevention and/or treatment of type 1 diabetes mellitus; or
c) in a method for the prevention and/or treatment of type 1 diabetes mellitus, wherein said composition is administered at an interval of from about 20 to about 36 days.

31-32. (canceled)

33. Use of i) an isolated polypeptide or peptide as defined in claim 2, ii) an isolated nucleic acid encoding the polypeptide or peptide, iii) an isolated peptide-MHC complex, wherein the peptide forming part of the complex is the polypeptide or peptide, iv) an isolated binding molecule capable of specifically binding the peptide-MHC complex or the polypeptide or peptide, or v) a polyclonal antiserum raised and/or directed against the polypeptide or peptide
a) for drug targeting;
b) for isolation, purification and/or quantification of a binding partner or a cell comprising a binding partner;
c) in in vitro and in vivo imaging;
d) in the diagnosis of type 1 diabetes mellitus; or
e) in detecting β-cell stress.

34-37. (canceled)

38. Use of i) an isolated polypeptide or peptide as defined in claim 2, ii) an isolated nucleic acid encoding the polypeptide or peptide, iii) an isolated peptide-MHC complex, wherein the peptide forming part of the complex is the polypeptide or peptide, iv) an isolated binding molecule capable of specifically binding the peptide-MHC complex or the polypeptide or peptide, v) a polyclonal antiserum raised and/or directed against the polypeptide or peptide, vi) a culture of tolerance promoting cells targeting the polypeptide or peptide, or vii) a composition comprising the isolated polypeptide or peptide
a) for the prevention and/or treatment of type 1 diabetes mellitus; or
b) as a medicament.

39-41. (canceled)

42. A method of treating a human subject having type 1 diabetes mellitus, or that is at risk of developing type 1 diabetes mellitus, said method comprising administering to said human subject an effective amount of i) an isolated polypeptide or peptide as defined in claim 2, ii) an isolated nucleic acid encoding the polypeptide or peptide, iii) an isolated peptide-MHC complex, wherein the peptide forming part of the complex is the polypeptide or peptide, iv) an isolated binding molecule capable of specifically binding the peptide-MHC complex or the polypeptide or peptide, v) a polyclonal antiserum raised and/or directed against the polypeptide or peptide, vi) a culture of tolerance promoting cells targeting the polypeptide or peptide, or vii) a composition comprising the isolated polypeptide or peptide.


SEQUENCE LISTING (12 sequences)

SEQ ID NO: 1 - RNA, 469 nt, Homo sapiens
agcccuccaggacaggcugcaucagaagaggccaucaagcagaucacuguccuucugccauggcccuguggaugcgccuccugccccugcuggcgcugcuggcccucuggggaccugacccagccgcagccuuugugaaccaacaccugugcggcucacaccugguggaagcucucuaccuagugugcggggaacgaggcuucuucuacacacccaagacccgccgggaggcagaggaccugcagguggggcagguggagcugggcgggggcccuggugcaggcagccugcagcccuuggcccuggaggggucccugcagaagcguggcauuguggaacaaugcuguaccagcaucugcucccucuaccagcuggagaacuacugcaacuagacgcagcccgcaggcagccccacacccgccgccuccugcaccgagagagauggaauaaagcccuugaaccagcaaaa

SEQ ID NO: 2 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 3 - protein, 43 aa, Homo sapiens
MLYQHLLPLPAGELLQLDAACRQPHTRRLLHRERWNKALEPAK

SEQ ID NO: 4 - protein, 43 aa, Homo sapiens
MLYQHLLPLPAGELLQLDAARRQPPTRRLLHRERWNKALEPAK

SEQ ID NO: 5 - protein, 95 aa, Homo sapiens
MALWMRLLPLLALLALWGPDPAAAFVNQHLCGSHLVEALYLVCGERGFFYTPKTRREAEDLQAGELLQLDAARRQPPTRRLLHRERWNKALEPAK

SEQ ID NO: 6 - protein, 18 aa, Homo sapiens
TRREAEDLQAGELLQLDA

SEQ ID NO: 7 - protein, 14 aa, Homo sapiens
LHRERWNKALEPAK

SEQ ID NO: 8 - protein, 9 aa, Homo sapiens
MLYQHLLPL

SEQ ID NO: 9 - protein, 9 aa, Homo sapiens
PTRRLLHRE

SEQ ID NO: 10 - DNA, 17 bp, Artificial Sequence
  Primer used to clone PPI into pJET2.1/blunt vector (generation of pJET1.2 - PPI and pJET1.2 - DRiP)
agccctccaggacaggc

SEQ ID NO: 11 - DNA, 20 bp, Artificial Sequence
  Reverse primer used to clone PPI into pJET2.1/blunt vector (generation of pJET1.2 - PPI)
gttgcagtagttctccagct

SEQ ID NO: 12 - DNA, 24 bp, Artificial Sequence
  Reverse primer used to clone PPI into pJET2.1/blunt vector (generation of pJET1.2 - DRiP)
ttttgctggttcaagggctttatt